(19) United States
(12) Patent Application Publication
(10) Pub. No.: US 2012/0121719 A1
(43) Pub. Date: May 17, 2012

(54) METHODS OF MAKING AND USING THREE-DIMENSIONAL EXTRACELLULAR MATRICES

(75) Inventors: Patrick A. Tresco, Sandy, UT (US); Jeffrey Collins Wolchok, Salt Lake City, UT (US)

(73) Assignee: UNIVERSITY OF UTAH RESEARCH FOUNDATION, Salt Lake City, UT (US)

(21) Appl. No.: 13/265,717

(22) PCT Filed: Apr. 22, 2009
(86) PCT No.: PCT/US09/41433

§ 371 Date: Feb. 2, 2012

(51) Int. Cl. A61K 35/12; A61P 17/00; A61P 25/00; A61P 19/00; A61P 9/00

(52) U.S. Cl. 424/520

(57) ABSTRACT

Three-dimensional extracellular matrices lacking synthetic scaffolds are provided. Methods of making the three-dimensional extracellular matrices and methods of using the three-dimensional extracellular matrices are also provided.

### STATEMENT REGARDING FEDERAL FUNDING

This invention was made with government support under National Institutes of Health grant numbers R01 NS046770 to Tresco. The government has certain rights in the invention.

### BACKGROUND

Tissue engineering strategies have commonly focused on the use of biodegradable synthetic polymers as supports for cell growth and tissue accumulation. Using this approach the formation of several tissues has been demonstrated, yet the development of commercially and clinically successful engineered tissues remains elusive. A detrimental host response to synthetic materials has blocked development of engineered tissue-based treatments. Biological materials harvested from original sources should be considered. Typically harvested from animal sources, biological materials have a level of bio-complexity and biocompatibility that synthetic materials do not possess. However use of tissues derived from animal sources also carries risks of infection and a detrimental host response directed against the tissues.

### SUMMARY OF THE INVENTION

Methods of preparing a three-dimensional extracellular matrix are provided herein. In one aspect, the methods include preparing a three-dimensional scaffold comprising a dissolvable polymer and adding cells to the three-dimensional scaffold to create a cellularized scaffold. The cellularized scaffold is cultured to allow production of extracellular matrix; and then treated with a solvent capable of dissolving the dissolvable polymer to produce the three-dimensional extracellular matrix. The three dimensional extracellular matrix made by this method is also provided.

In another aspect, a three-dimensional extracellular matrix is made by preparing a three-dimensional scaffold comprising a dissolvable polymer and contacting the scaffold with a liquid extracellular matrix composition to form a scaffold-extracellular matrix mixture. The resulting mixture is then treated to solidify the extracellular matrix to produce a scaffold-extracellular matrix composition. The scaffold-extracellular matrix composition is then contacted with a solvent capable of dissolving the dissolvable polymer to produce a three-dimensional extracellular matrix. The three dimensional extracellular matrix made by this method is also provided.

In another aspect, methods of generating a three-dimensional extracellular matrix are provided. The methods include implanting a three-dimensional scaffold comprising a dissolvable polymer in a subject to produce a cellularized scaffold. The cellularized scaffold is harvested from the subject and then treated with a solvent capable of dissolving the dissolvable polymer to produce the three-dimensional extracellular matrix. The three dimensional extracellular matrix made by this method is also provided.

In yet another aspect, methods of generating a tissue in a subject are provided. These methods include implanting the three-dimensional extracellular matrix in the subject.

In still another aspect, methods of treating a subject in need of a tissue implant are provided. The methods include administering the three dimensional extracellular matrix to the subject.

In yet another aspect, methods of treating a subject are provided. The methods include combining the three-dimensional extracellular matrix described above with at least one cell to produce a cell-extracellular matrix composition. This composition is then implanted in the subject. The implanted cell-extracellualar matrix is capable of treating the subject.

### BRIEF DESCRIPTION OF THE DRAWINGS

FIG. 1A is an SEM image of open celled polymer foam. FIG. 1B is a photograph showing foams that were sectioned into strips and attached to Mylar mounts using a UV curable adhesive.

FIG. 2A is a photograph of modified T-flask capable of housing four mounted samples. FIG. 2B is a photograph showing that samples are accessed through an adhesive backed lid. FIG. 2C is a diagram showing that mounted samples are suspended in the flask to ensure that they are completely surrounded by media.

FIG. 3A is a diagram showing removal of the PU foam by soaking samples in DMAC for 72 hours. Representative images of pre and post DMAC treated constructs (B and C respectively). Cell free control foams were completely dissolved (C-left panel), however substantial material remained following DMAC treatment of LF seeded samples (C-right panel). LF seeded samples, yielded 48 mg of material for every gram of seeded PU foam (D).

FIG. 4 is a graph showing that serial digestion resulted in complete degradation of extracted material. Following DMAC removal of the PU foam (step 1), chondroitinase ABC and collagenase IV treatment removed 5% and 80% of the extracted material respectively (steps 2 & 3). The remaining 15% was digested following SDS and DNAse treatment (step 4).

FIG. 5 is a set of graphs showing representative stress strain curve (A), ultimate strength (B) and elastic modulus (C) values (mean±SEM, n=3) for materials extracted from LF seeded foams following 3 weeks in culture.

FIG. 6A is a photograph of lyophilized material extracted from LF seeded foams following DMAC treatment. FIG. 6B is H & E staining of an extracted material thin section.

FIG. 7 is a set of micrographs of SEM (A & B) & TEM (C & D) images of extracted material. TEM imaging of material cross sections revealed a porous interior network surrounded by a thick outer layer (C arrow). Fibrillar structures indicative of extracellular matrix could be seen in both the SEM (B) and TEM (D arrow) images.

FIG. 8 is a photograph of Calcein AM viability staining of extracted material reseeded with LFs. Viable cells were well distributed throughout the material following 48 hours in culture.

FIG. 9 is a set of photographs of DAPI (A) and vimentin (B) staining of control and decellularized samples imaged side by side for comparison. When stained with DAPI nuclei are seen as blue spots. Vimentin, a filamentous intracellular cytoskeleton protein, is stained red. Nuclei and vimentin staining is evident in control samples. The SDS/nuclease decellularization process effectively eliminated both nuclear and vimentin staining

FIG. 10 is a set of micrographs of thin section H&E staining of control (A) and decellularized (B) material sections. Nuclei are seen as black/purple spots in control samples. Similar nuclei staining is not seen in decellularized samples (Scale bar=100 μm).

FIG. 11A is a graph showing the stress-strain curves for cytokine conditioned, control, and unseeded naïve PU foams (from top moving down on graph is TGF-β1 treated, HGF treated, control and unseeded naïve). FIG. 11B is a graph of the elastic modulus (mean±SEM, n=4), which was significantly increased over control values for both HGF and TGF-β1 conditioned samples.

FIG. 12A is a photograph showing representative DMAC extracted and lyophilized materials from control and cytokine conditioned samples. FIG. 12B is a bar graph showing that the material yield (mean±SEM, n=4) was significantly increased following three weeks of both HGF and TGF-β1 cytokine conditioning.

FIG. 13 is a graph showing the collagen yield results (mean±SEM, n=4) for control and cytokine conditioned samples. Both HGF and TGF-β1 conditioning resulted in significant yield increases. (ANOVA, p<0.05)

FIG. 14 is a graph showing the GAG yield (mean±SEM, n=4) results for control and cytokine conditioned samples. Both HGF and TGF-β1 conditioning resulted in significant yield increases. (ANOVA, p<0.05)

FIG. 15 is a set of micrographs showing representative sections following H&E staining of control (A), HGF (B) and TGF-β1 (C) treated thin sections. (Scale bar=100 μm)

FIG. 16 is a photograph showing dual mode bioreactor with strain linkage and stepper motor, vocal coil actuator, and modified t-flask with cell seeded PU foam samples.

FIG. 17 is a schematic showing the strain and vibration conditioning protocol which was used:

FIG. 18A is a graph showing representative stress strain curves for static control, strain, and dual mode conditioned samples. FIG. 18B is a graph showing mechanical conditioning significantly increased sample elastic modulus values (mean±SEM, n=4). (* p<0.05 compared to control)

FIG. 19A-C is a set of photographs showing DMAC extracted and lyophilized control (A), strain (B), and dual mode (C) conditioned samples. FIG. 19D is a graph showing that as compared to static controls yield (mean±SEM, n=4) was significantly increased for both stain and dual mode conditioned samples (* p<0.05 compared to control). Furthermore, dual mode yield increases were statistically significant when compared to strain conditioned values.

FIG. 20 is a graph showing GAG yield values (mean±SEM, n=4) for control and conditioned samples. Dual mode yield values were significantly increased over strain and static control values (*p<0.05 compare to control).

FIG. 21 is a set of micrographs showing representative H&E stained static (A), strain (B), and dual mode (C) conditioned samples. For strain conditioned samples the extracted material showed evidence of alignment in the direction perpendicular to strain. Arrow indicates strain direction. (Scale bar=100 μm)

FIG. 22 is a set of photographs showing the high frequency vibrational bioreactor in the culture plate configuration (A) showing 1) the voice coil actuator 2) 6 well culture plate, and 3) base. With a configuration change, the bioreactor can accommodate a custom designed culture well (B).

FIG. 23 is a photograph of a culture well with press fit polyurethane porous sheet forming the base as shown from the top, bottom, and cross section views. Scale bar=1 cm.

FIG. 24 shows the characterization of the vibrational bioreactor. The bright traces produced by fluorescent microspheres adsorbed to six well or single well dishes

(A) were used to determine vibration amplitudes with the aid of image analysis software (B). FIG. 24C is a graph showing the vibrational amplitude as a function of frequency as measured over a range of 100 to 200 Hz for both the culture plate and culture well configuration. The maximum acceleration was calculated from the measured displacement data and averaged across frequency for each configuration (inset).

FIG. 25 is a set of graphs showing TGF-β1 and MCP-1 cytokine levels for static control and vibration conditioned laryngeal cell cultures. Fibroblasts were conditioned for two hours distributed evenly over a single six hour conditioning period. Media was collected the following day, 24 hours after the start of conditioning (N=4). *=p<0.05

FIG. 26 is a set of confocal images of matrix protein accumulation and distribution for three day control and vibrated scaffolds. Cell seeded scaffolds were stained for the presence of collagen type I and insoluble cellular fibronectin.

FIG. 27 is a schematic diagram showing the average fluorescence intensity values for three day control and vibrated scaffolds expressed as a percentage of control. The immunoreactivity to both cellular fibronectin and collagen type I for vibrated scaffolds was significantly increased over static controls (N=4). *=p<0.05

FIG. 28 is a set of graphs showing representative stress-strain curves for vibration conditioned and static control samples. From the stress strain curves the elastic modulus was calculated within both low (5-25%) and high strain regions (30-50%). * =p<0.05 relative to static controls. (N=3 samples/group)

FIG. 29 is a set of micrographs of lyophilized tissue sample isolated from long term vibration conditioned scaffold (A and B) and H & E staining of sectioned tissue illustrating cell and matrix accumulation within long term conditioned samples (C and D).

FIG. 30 is a set of graphs showing representative stress-strain curves for LF, hMSC, and unseeded (naïve) samples (A). While LF seeded sample elasticity significantly increased following three weeks in culture, the hMSC sample elastic module was not significantly different from naïve samples (B) (n=3/group). *=P<0.05 compared to naïve controls.

FIG. 31A-B is a set of photographs of lyophilized material extracted from hMSC seeded foams (A). H&E staining revealed a structure similar to material extracted from LF seeded foams (B). FIGS. 31C and D are graphs showing that although the yield from hMSC seeded foams was similar to LF seeded foams (C), hMSC extracted materials contained little collagen (D). Data displayed as mean±SEM (n=3).

### DETAILED DESCRIPTION

Three-dimensional extracellular matrix (3-D ECM) compositions are provided. Also provided are methods of making and methods of using the 3-D ECMs. The method of making a 3-D ECM includes preparing a 3-D scaffold comprising a dissolvable polymer. In the Examples, polyurethane (PU) foam was used as the scaffold. As those of skill in the art will appreciate a wide variety of scaffold materials could be used in the methods. For example, a wide range of phase inverted polymeric materials whose structure consists of chain entanglements, which can be overcome with weak aprotic solvents could be envisioned. This includes most of the biodegradable polymers including PLA and PGA as well other accepted polymeric biomaterials including PAN/PVC and polysulphone that are not commonly considered for tissue engineering applications. Suitably, a thermoplastic polymer is used to make the scaffold, suitably the scaffold is made of a biomedical grade material. Suitably, biodegradable materials such as poly(ε-caprolactone) (PCL), poly(L-lactic acid) (PLA), or poly(lactic-co-glycolic acid) (PLGA) are used.

The scaffold can be formed into a 3-D structure using a wide variety of techniques. In the Examples, a precipitation technique was used to prepare PU foams. A variety of methods capable of creating porous polymer scaffolds including gas foaming, particle leaching, hollow fiber extrusion, electrospinning, phase separation and spray phase inversion could be used to create solvent degradable scaffolds. The scaffolds used herein have an open celled structure, but other structures may be used. The open celled structure may allow penetration of cells and vessels into the open spaces to facilitate integration of the engineered tissues after implantation into a subject and facilitate transport during cultivation ex vivo.

After the 3-D scaffold is prepared, cells are added to the 3-D scaffold to create a cellularized scaffold and the cellularized scaffold is cultured to allow production of ECM. Cell types useful in the methods, include but are not limited to, anchorage-dependent cells, fibroblasts, stem cells, mesenchymal stem cells, osteoblasts, chondroblasts, tenocytes, and myocytes. Any cell type or combination of cell types capable of producing ECM or inducing production of ECM may be used. In the Examples, fibroblasts, specifically human laryngeal fibroblasts (LG) were used. Other fibroblasts specific to certain tissue types may be suitable as well, including e.g., vascular fibroblasts. Various stem or precursor cells may be included as well, such as retinal or glial precursors. Use of the method with other anchorage dependant cell types may create additional ECM materials with potentially distinct properties and applications. The results presented in Example 5 support this distinction. For example, materials extracted from astrocyte seeded foams could have several applications within the field of neural tissue engineering including bridging devices, dural substitutes, or scaffolding for stem cell transplantation which are distinct from uses of ECM materials from laryngeal fibroblast seeded foams which may have applications in repair of the larynx or voice box.

Those of skill in the art will appreciate that the cell type or combination of cell types used will effect the resulting ECM composition and characteristics. Dermal or soft tissue fibroblasts, like the ones described in the examples, would deposit an ECM with properties similar to several tissues including laryngeal but also abdominal (used during hernia repair), vaginal (vaginal prolapse repair), and subcutaneous tissues (plastic or reconstructive surgery). Other cells could be used to create more specific tissues including cardiac (cardiac myocytes), vascular (vascular fibroblasts), retinal (retinal precursors, nervous (glial precursors), cartilage (chondrocytes), and bone (osteoblasts). Additionally, non-human animal cells could be used to create materials with research or veterinary applications.

The cellularized scaffold may be maintained in culture for a wide range of time and treated in various ways in culture to affect the production and composition of the ECM. In the Examples, the cellularized scaffolds were cultured for three weeks. The scaffolds may be cultured for as little as one week, two weeks, three weeks, or one month. Alternatively, very long culture periods could be useful for certain applications, such as tendon or ligament replacement where the ECM must be quite strong. Culture periods of two months, three months, four months, five months, six months or even one year or more may be used.

During the culturing period the cellularized scaffold may be treated to alter the composition and functional characteristics of the ECM. In one embodiment, the cellularized scaffold is contacted with an agent capable of altering the ECM. The agent may be a cytokine, a growth factor or a cell. In the Examples, transforming growth factor-β1 (TGF-β1) and hepatocyte growth factor (HGF) were added to cellularized scaffolds. Contact with these agents was shown to result in an increase in material yield and elastic modulus in the resulting 3-D ECMs. The Examples demonstrate that the increased yield was partially due to increased collagen and GAG production by treated cellularized scaffolds as compared to untreated scaffolds. In particular, TGF-β1 treatment increased collagen yield and HGF increased yields of GAG. Those skilled in the art will appreciate that other cytokines or growth factors may also be used or cells producing these factors could be added to the cellularized scaffold to produce 3-D ECM with distinct compositions and characteristics.

In another embodiment which may be practiced in conjunction with the embodiments described above, the cellularized scaffolds are subjected to mechanical or physical stress. Those of skill in the art will appreciate that a variety of physical or mechanical stressors could be utilized, including but not limited to cyclic strain, vibrational stress, cyclic compressing and fluid flow In the Examples, cyclic strain and high frequency vibrational stress were used as mechanical stressors. The Examples were focused on laryngeal tissue and thus cyclic strain and vibrational stress are appropriate conditioners for the tissue based on the physiological ranges for vocal strain. One of skill in the art will appreciate that other tissue types may require different tissue-appropriate stressors to develop the appropriate ECM composition and physical characteristics. Cyclic strain may be performed in any axis and encompasses a wide range of strains including, but not limited to, 2%, 5%, 7%, 10%, 12%, 15%, 17%, 20% or more strain. The strain may be repeated at a wide range of frequencies. In the Examples, a 10% strain at a frequency of 0.25 Hz was used. The frequency may range from about 0.1 Hz to about 1 Hz or more suitably from 0.1 Hz to 0.7 Hz, suitably from 0.2 Hz to 0.5 Hz suitably from 0.25 Hz to 0.4 Hz. The vibrational stress may also be performed at a variety of frequencies. In the Examples, a frequency of 100 Hz was used. Frequencies from about 10 Hz to 1000 Hz or more may be used suitably the frequency is from about 10 Hz to 500 Hz, suitably from about 50 Hz to about 250 Hz, suitably from 75 Hz to 150 Hz. The cellularized scaffolds may be exposed to the stress for any portion of the culture period. Supra-physiological stress may be used to produce high strength ECM for certain uses.

In the Examples, the cellularized scaffolds were conditioned for eight hours a day for three weeks. The strain may be applied for as little as 2 hours a day or up to 24 hours a day and be applied one time or during the entire culture period. The

Examples demonstrate that exposure of the cellularized scaffold to the combination of cyclic and vibrational strain resulted in production of a 3-D ECM having remarkably increased GAG production, material yield, and elastic modulus. Cyclic strain alone induced alignment of the tissue which was eliminated by the addition of the vibrational stress. High frequency vibration increased GAG production, yield and elastic modulus by about 50% over cyclic strain alone.

After culturing is complete, the cellularized scaffold is treated with a solvent capable of dissolving the dissolvable polymer to produce the 3-D ECM. The solvents are generally weak aprotic solvents and as one skilled in the art will appreciate the solvent will be matched to the polymer used to make the scaffold. Suitably, the solvent is selected from dimethyl acetamide, dimethyl formamide, dimethyl sulfoxide, chloroform, tetrahydrofuran, acetone, or ethyl acetate. Other suitable solvents capable of dissolving the polymer while leaving the ECM may also be used.

The 3-D ECM may be further treated to remove any cells or cell debris to create a decellularized ECM. Those skilled in the art will appreciate that several protocols for decellularizing ECM are available including Mirsadraee S Et al., Development and characterization of an acellular human pericardial matrix for tissue engineering, Tissue Engineering, 2006 April; 12(4):763-73; Rosario et al., Decellularization and sterilization of porcine urinary bladder matrix for tissue engineering in the lower urinary tract, Regen Med, 2008 March; 3(2):145-56; and Gilbert T W, Sellaro T L, Badylak S F. Decullarization of Tissues and Organs. Biomaterials. 2006 July; 27(19):3675-83 each of which is incorporated herein by reference in its entirety. A decellularized ECM may be useful because allogeneic cells could be used to generate the ECM and then prior to using the ECM, the cells would be removed to avoid a deleterious immune response.

Thus a 3-D ECM and a decellularized ECM are provided herein. These tissues may contain a wide array of compositions depending on the cell type used and the culture conditions including the introduction of mechanical stimulation as described above. The ECM may contain collagen, glycosaminoglycans (GAG), fibronectin, laminin, elastin, hyaluronate, and many other constituents. The relative amounts of these constituents may vary.

One of the ongoing challenges when engineering successful replacement tissue is achieving mechanical properties that approximate native tissue values. The Examples demonstrate the development of an engineered replacement for damaged vocal tissue. Reported values for vocal tissue elastic modulus range from 30 to 400 kPa. The elastic modulus for the extracted material produced in the Examples is similar to measured vocal tissue values. Other load bearing tissues, including tendons and ligaments, have mechanical properties that are several orders of magnitude higher (kPa versus GPa). Those skilled in the art will envision methods to increase the mechanical properties of the engineered 3-D ECMs provided herein to produce tendons/ligaments. For example, it may be possible to increase the strength and stiffness of the 3-D ECM by incorporating various pro-fibrogenic stimuli into a conditioning program during growth in culture as demonstrated by conditioning with HGF and TGF-β1. It is well established that the mechanical environment that cells are subjected to can be used to increase ECM production as demonstrated herein.

In an alternative embodiment, a 3-D ECM may be prepared by preparing a 3-D scaffold as described above and contacting the scaffold with a liquid ECM composition to form a scaffold-ECM mixture. Such liquid ECM components are commercially available or could be produced by methods available to those skilled in the art. The scaffold-ECM mixture is then treated to solidify the ECM and produce a scaffold-ECM composition. The scaffold-ECM composition may then be treated with a solvent capable of dissolving the dissolvable polymer to make a 3-D ECM product. Cells may be added to the 3-D ECM to form a cellularized 3-D ECM. The cells may further modify the ECM. The ECM may be decellurlarized prior to implantation or implanted with cells.

In another alternative embodiment, a 3-D ECM may be generated by implanting a 3-D scaffold comprising a dissolvable polymer in a subject to produce a cellularized scaffold. After a period of incubation or culture in the subject the cellularized scaffold is harvested from the subject. The cellularized scaffold is treated with a solvent capable of dissolving the dissolvable polymer to produce the 3-D ECM. The subject may be any mammalian subject, suitably the subject is a human.

The 3-D ECMs described herein may be used in a variety of methods, including but not limited to methods of generating a tissue in a subject and methods of treating subjects in need of an implant. Alternatively, the 3D ECMs may be used as a vehicle to deliver cells to a subject. For example the 3-D ECM may be used to deliver transplanted cells of tissues to a subject. The methods include implanting the 3-D ECM produced by the methods described above into the subject. The 3-D ECM may be implanted by any method, including those known to those of skill in the art such as surgical implantation or injection through a syringe, needle or other suitable catheter.

The 3-D ECMs may be useful to repair tissues. For example, the 3-D ECMs may be used to repair larynx, tendon, voice box, ligament, bone, cartilage, soft tissue, nervous tissue, skin, cardiac tissue, and vascular tissue. Alternatively, the 3-D ECMs may be used as a support for transplantation of cells, such as stem cells, or as a carrier of growth factors or other factors capable of eliciting/supporting growth and development of new tissue, such as nervous or dental tissues. The 3-D ECM may also serve as a vehicle for sustained or gradient-based delivery of an agent, such as a polypeptide. In this embodiment, the 3-D ECM may be combined with cells or agents such as polypeptides, e.g. cytokines or growth factors, to affect metabolic, growth or endocrine functions near the implant site. Alternatively, the 3-D ECM may be implanted into a tissue cavity in the host tissue as a preparatory step to the transplantation of cells or tissues in a subject. For example, the 3-D ECM may be used to promote vascularization, or to prepare a site for later transplantation or nerve regeneration.

The Examples included herein are meant to be illustrative and not to limit the claims.

### EXAMPLES

### Example 1

### Engineering a Human Cell-Derived ECM Material Methods

Polyurethane Foam Preparation

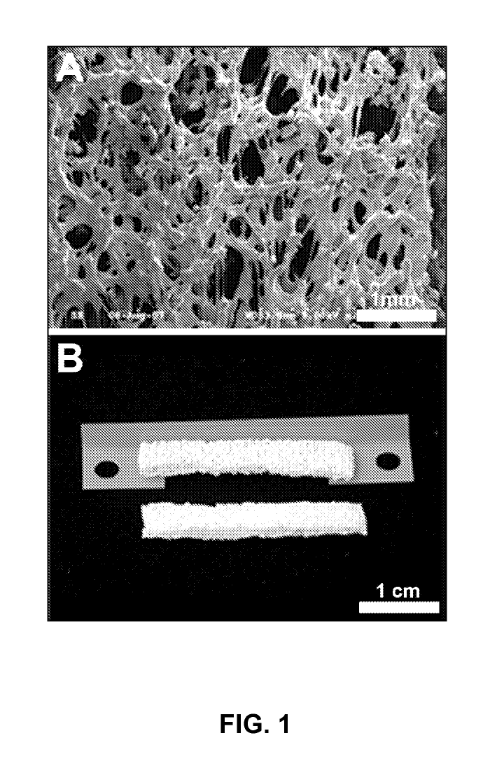

Polyurethane pellets (4 g) (Tecoflex SG-8, Thermedics) were dissolved in dimethylacetamide (40 ml) (DMAC) overnight at 60° C. Pluronic 10R5 (20 ml) (BASF) was added and the solution thoroughly mixed. The polymer solution was cooled to 46° C., pored into polymer molds, further cooled for 2.5 minutes in a dry-ice/ethanol bath, and precipitated overnight in a DI water bath. The resulting foams have an interconnected pore network, high flexibility, and can be molded into a variety of geometries (FIG. 1). Scaffolds were separated from the molds, rinsed in DI water for 48 hours, frozen to −80° C., and lyophilized. Lyophilized scaffolds were sectioned into strips (30 mm×10 mm×2 mm) and attached to Mylar mounts using medical grade UV curable adhesive (MD 1180-M, Dymax, Torrington, Conn.). Prepared scaffolds were ethylene oxide sterilized, soaked for 20 minutes in 70% ethanol to promote wetting, rinsed in DI, and soaked overnight in a fibronectin solution (20 μg/ml in DI). The process has been described previously.

Seeding and Culturing

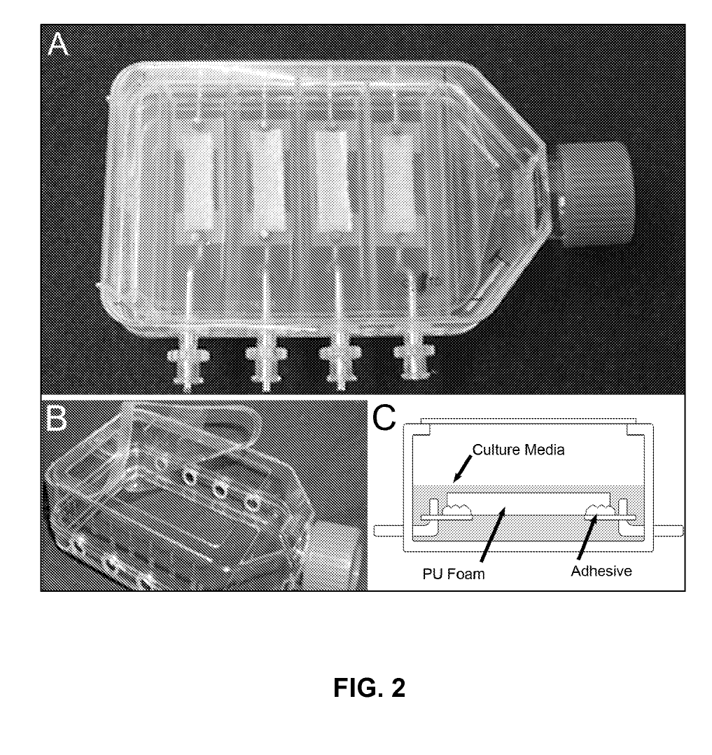

Human laryngeal fibroblasts (LFs) originally obtained during biopsy, were used for all experiments. Prior to testing frozen cells were thawed, plated in T-175 flasks, and grown to confluency in DMEM/F12 (Gibco) with 10% FBS (Gibco) and 25 ug/ml gentamicin (Sigma). Confluent flasks were disassociated with 0.25% trypsin and 1 mM EDTA, centrifuged, and resuspended in DMEM/F12. Cells were counted with the aid of a hemocytometer and seeded onto scaffolds at a density of 2 million cells/foam. Samples were cultured for three weeks in a modified T-flask (FIG. 2) and maintained in a growth medium consisting of DMEM F12 supplemented with 10% FBS, gentamicin, and 1 mM ascorbic acid. Non-seeded foams were maintained as controls. Media was changed every 2-3 days for three weeks.

Material Extraction and Serial Digestion

At the completion of the culture period samples (n=4) were rinsed in DI, frozen to −80° C. and lyophilized. Lyophilized samples were weighed and then soaked in the solvent DMAC for 72 hours at 37° C. to remove the PU foam. The extracted material was rinsed three times in DI and lyophilized. Yield was calculated relative to initial PU foam weight.

$$Yield = \frac{D M A C Extracted Material Weight ( mg )}{P U Foam Weight ( g )}$$

To evaluate material composition a subset of DMAC extracted samples (n=3) were serially digested beginning with 1 unit/ml chondroitinase ABC (Acorda Therapeutics), followed by 0.5% collagenase IV (Worthington), and finishing with a 0.1% SDS (Genemate), and 0.2 mg/ml DNAse (Sigma) solution. Samples were soaked in each respective digestion solution for 24 hours at 37° C. with agitation, rinsed in DI, lyophilized, and weighed between steps.

Mechanical Testing

DMAC extracted material properties were measured with the aid of a uni-axial tensile tester. Hydrated (PBS, pH=7.4) samples (n=3) were placed into custom grips, and deformed at a constant strain rate of 10%/s until failure using a material testing system incorporating a 1 kg load cell with a resolution of 0.015 g. Custom control software was used to control deformation rate and record force/elongation data. Prior to testing, samples were imaged and initial dimensions were measured from the digital images. For each scaffold, engineering stress versus strain curves were generated from load, and elongation data. Elongation was determined from grip displacement. From each curve the tan modulus and ultimate strength was calculated. The tan modulus was calculated from a linear curve fit to the stress-strain curve. The ultimate strength was calculated as the maximum engineering stress attained by the sample prior to failure.

Imaging

Sections of representative DMAC extracted samples were dehydrated in acetone, resin imbedded (Technovit 8100, Heraeus, Germany), thin sectioned (5 μm) with the aid of a microtome (Jung R M 2035, Leica, Germany) and mounted on microscopic slides. Sections were stained with hemotoxylin and eosin (H&E) to visualize sample architecture. A sample was also prepared for scanning electron and transmission electron microscopy (SEM & TEM).

Cell Toxicity

DMAC extracted materials and control PU foams were sterilized using a 20 minute 70% ethanol soak. Samples (n=2/group) were reseeded with LFs at a density of 50K cells per sample (approximate size 2×2×2mm). Samples were maintained in 10% FBS supplemented media for 48 hours. At the end of the 48 hour period cell viability was assessed with calcein AM (Invitrogen, Carlsbad, Calif.).

Decellularization

Samples (n=3) were decellularized using a protocol previously used to decellularized human pericardial tissue (Mirsadraee, S., et al., Development and characterization of an acellular human pericardial matrix for tissue engineering. Tissue Eng, 2006. 12(4): p. 763-73). Briefly, samples were rinsed for 90 min in Tris-HCL (10 mM, pH8.0) with 1% EDTA and 10 KIU/m1 aprotinin 4° C. with agitation. Sections were then soaked in 0.1% SDS in Tris-HCL buffer for 24 hours at room temperature with agitation. Tissues were then incubated for 3 h in reaction buffer containing 50 U/mL deoxyribonuclease I and 1 U/mL ribonuclease A in 10 mM Tris-HCl (pH 7.5)) at 37° C. with agitation. To evaluate decellularization, treated and control samples were immunostained for vimentin using the appropriate primary (αhuman vimentin, mouse IgG1, 500:1, Sigma, MO) followed by fluorophore labeled secondary antibodies (αmouse IgG1, 500:1, Invitrogen, CA). Cells were counterstained with the nuclear dye DAPI. Decellularized samples underwent material extraction, material testing, and thin section imaging as previously described.

Data Analysis

A students t-test was used to compare decellularized material properties (yield, elastic modulus, and ultimate strength) with cellularized samples. A 0.05 level of significance was used for all comparisons. All data is displayed as the mean with standard error of the mean (SEM).

Results

Material Extraction and Digestion

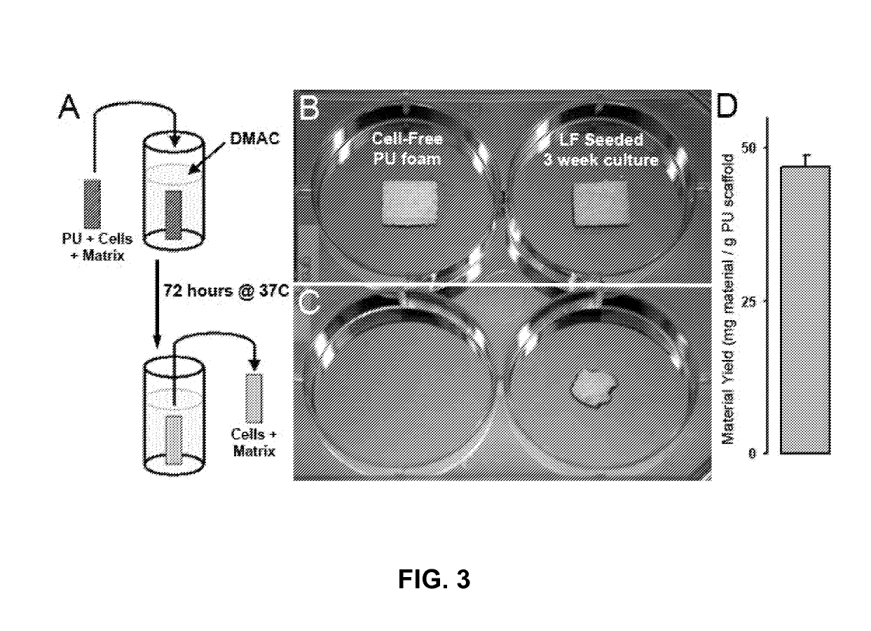

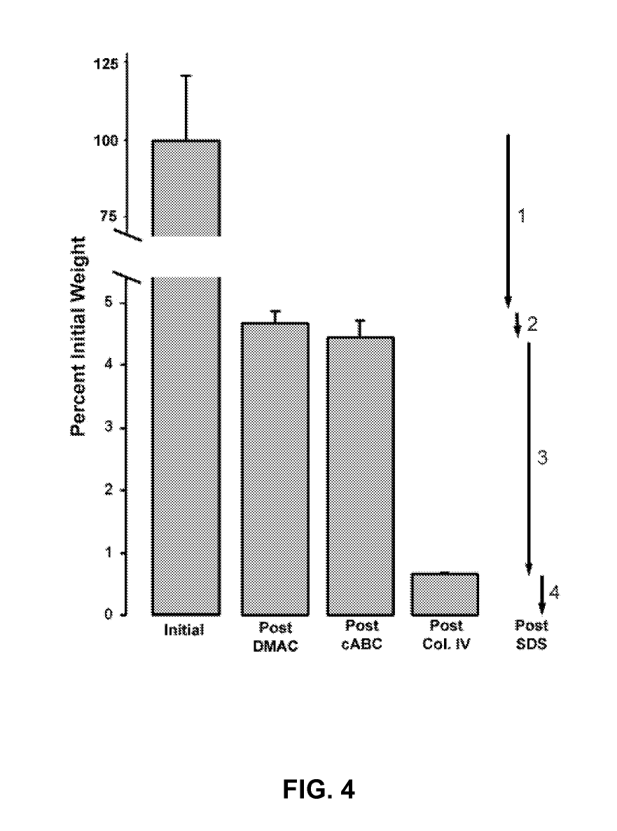

Substantial material was extracted from LF cultured foams following DMAC dissolution. Following a three-week culture period, samples yielded 48±2 mg of material for every gram of seeded PU foam (FIG. 3). Subsequent serial digestion resulted in complete degradation of the DMAC extracted material. Chondroitinase ABC and collagenase IV treatment removed 5% and 80% of the extracted material respectively. The remaining 15% was fully digested following SDS and DNAse treatment (FIG. 4). Residual PU foam was not observed following digestion.

Mechanical Testing

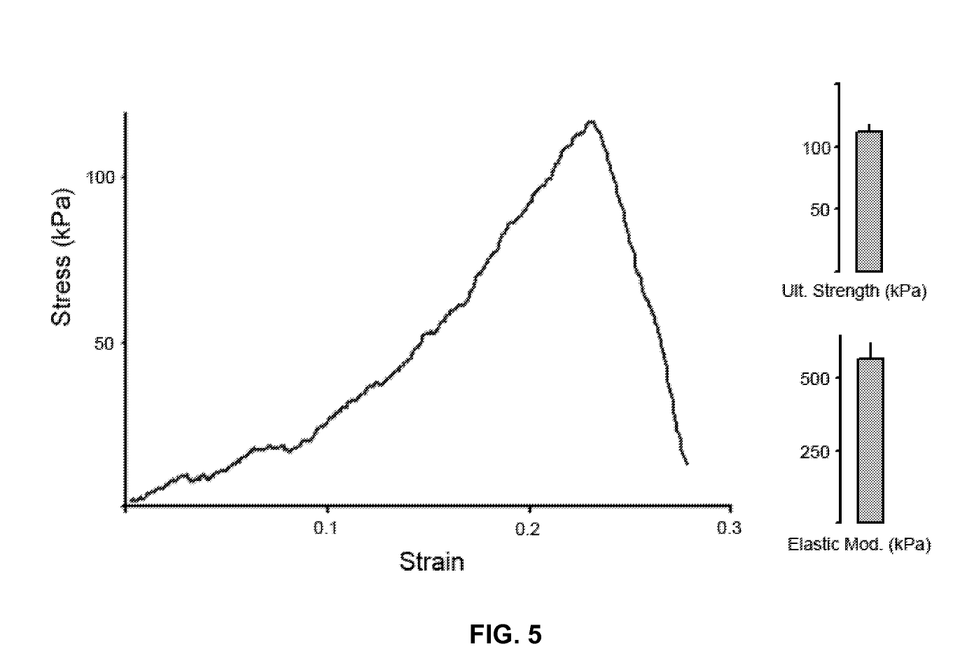

Stress-strain curves were generated for both cellularized and decellularized extracted material samples (FIG. 5). LF seeded sample stress-strain curves were characterized by a short toe-in region followed by a linear increase in stress extending out to failure at approximately 25%. Similar stress versus strain patterns were observed for each sample tested. The average ultimate strength was 115±1 kPa. The ultimate stress was attained just prior to failure for each sample tested.

To avoid the toe-in region, the tan modulus was determined for the stress-strain region extending from 5% strain to failure. The average tan modulus for the toe-in to failure region was 501±139 kPa

Imaging

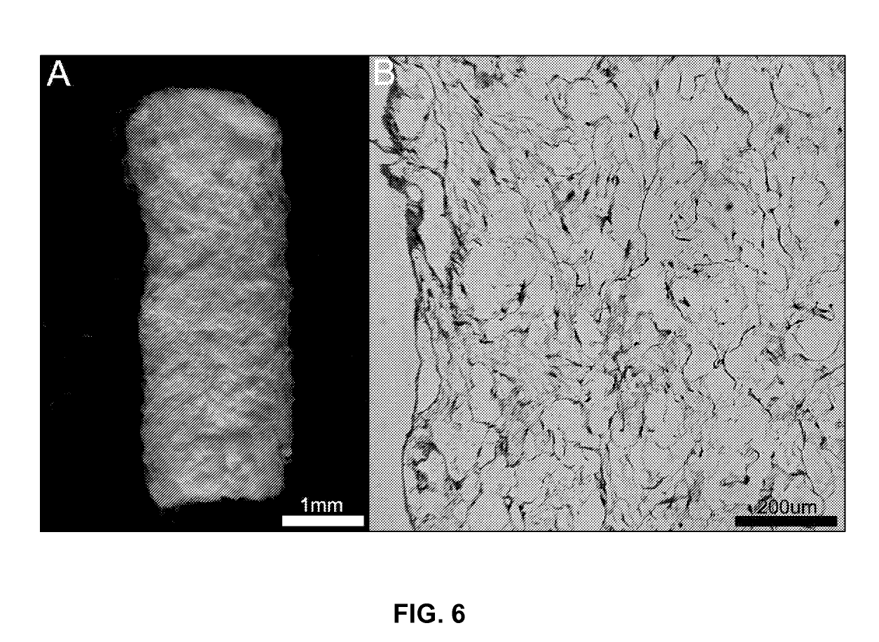

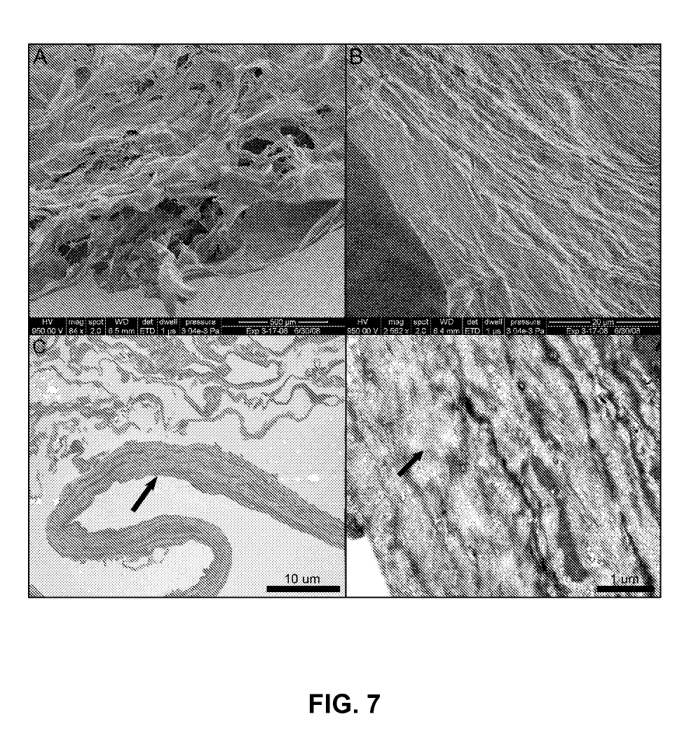

H&E staining of DMAC extracted sections revealed an interconnected porous material consisting of both cell bodies and accumulated ECM material (FIG. 6). PU foam remnants were not evident within the sections. TEM images revealed a fine interior network surrounded by a thicker outer layer. The rough topography and porous structure of the outer layer could be seen in SEM images. At higher magnifications, fibrillar structures indicative of extracellular matrix could be seen in both the SEM and TEM images (FIG. 7).

Cell Toxicity

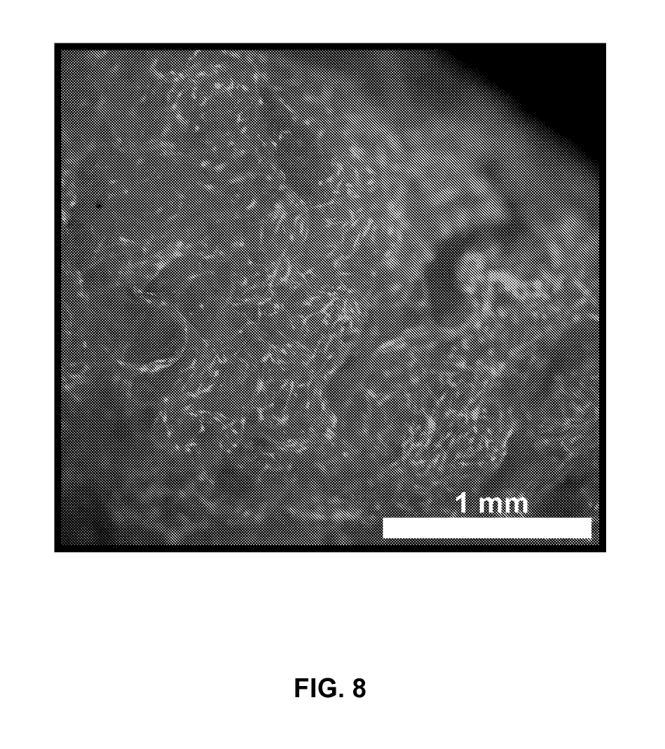

Viable cells were present and densely distributed across the surface and within the pores of reseeded samples (FIG. 8). Viable cell density within reseeded DMAC extracted materials was qualitatively similar to seeded PU foam controls

Decellularization

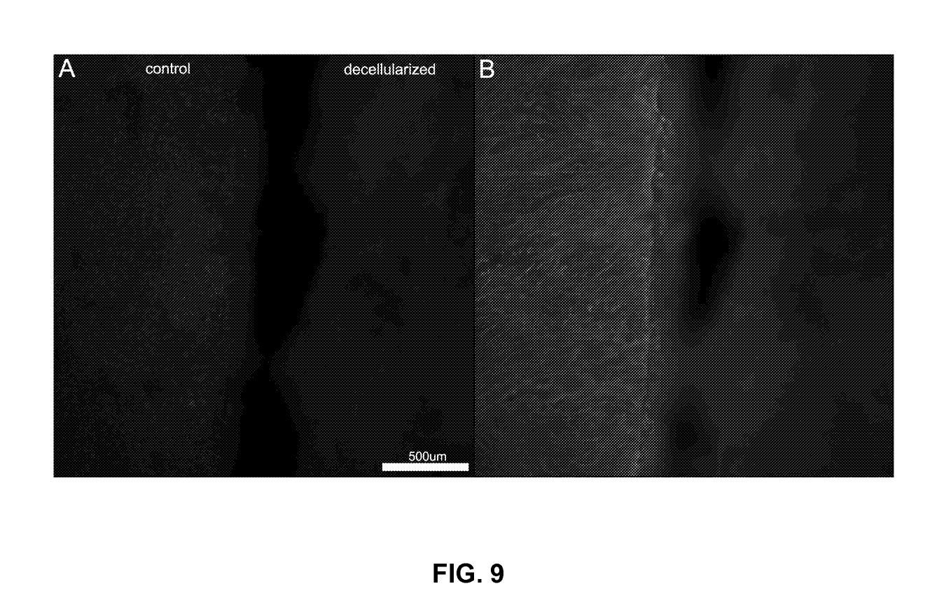

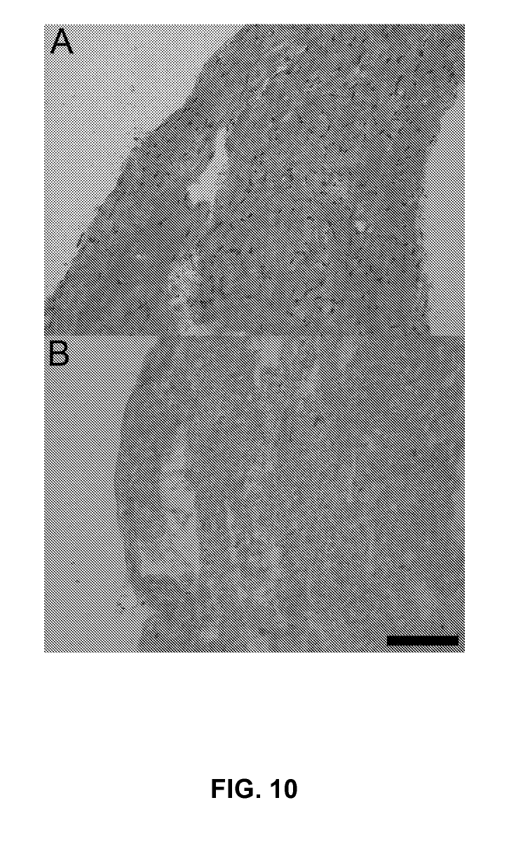

Vimentin and DAPI staining was apparent in cellularized controls. The immuno-reactivity to vimentin was markedly decreased in decellularized samples (FIG. 9). Similarly, DAPI staining of cell nuclei was absent within decellularized samples. DMAC material extraction from decellularized samples yielded approximately 20% less material than cellularized controls. While the yield was decreased, the gross thin section histological appearance was similar to cellularized controls (FIG. 10). The stress strain behavior of the material was affected by the decellularization process. The modulus of elasticity was reduced by 50%, from 507 kPa to 253±57 kPa,. The strain at failure also increased from 25% to 75%. However, the ultimate strength (136±32 kPa) was not significantly affected.

The above demonstrates in-vitro method to 1) culture cells and accumulate cell derived material within PU foams, and 2) extract the accumulated cell and ECM material from the foams using a solvent dissolution technique. We believe these extracted materials may be used as implant scaffolds and that this technique represents an altogether new approach to create biological scaffolds.

The material can be fabricated with thicknesses that go well beyond that of cell sheets and therefore could be used to repair larger tissue defects in a manner similar to SIS scaffolds. Lastly, the accumulation and extraction of biological material is accomplished in-vitro and therefore could potentially be scaled up to allow for commercial production.

Therefore, a method to extract a biological scaffold from an underlying foam support has been developed. The extracted material consists of human ECM and cell remnants, which support cellular attachment. The open architecture of these materials could provide a natural conduit for the ingrowth of vessels and nerves. Because the accumulated material is extracted from the polymer scaffold, the technique is not limited to the traditional biodegradable polymers, and we suspect any number of phase inverted polymer systems and materials could be used. The material has potential applications in several fields that are currently dominated by a variety of synthetic and natural materials. These include soft tissue augmentation, connective tissue repair, wound healing, nerve guidance, as well as components of various implantable biomedical devices.

### Example 2

### Use of Cytokines to Improve the ECM

Methods

Substrate Preparation:

Polyurethane pellets (4 g) (Tecoflex SG-8, Thermedics) were dissolved in dimethylacetamide (40 ml) (DMAC) overnight at 60° C. Pluronic 10R5 (20 ml) (BASF) was added and the solution thoroughly mixed. The polymer solution is cooled to 46° C., quenched in a ethanol/dry ice bath for 2.5 minutes, and precipitated overnight in DI water. Substrates are separated from the molds, rinsed in DI water for 48 hours, frozen to −80° C., and lyophilized. Lyophilized scaffolds are sectioned (30 mm×10 mm×2mm) and attached to Mylar mounts using medical grade UV curable adhesive (Dymax, Torrington, Conn.). Prepared scaffolds were ethylene oxide sterilized at the University of Utah Hospital sterilization facility. To facilitate cellular attachment, scaffolds were soaked overnight in a fibronectin solution (20 ug/ml) (Invitrogen, Carlsbad, Calif.).

Cell Culture:

For all treatment and control conditions, human laryngeal fibroblasts (LFs) were used. Cells were originally isolated from a sample of human tracheal tissue obtained by biopsy from a patient undergoing surgery. Frozen larygenal fibroblasts were thawed, plated in T-175 flasks, and grown to confluency in DMEM/F12 with 10% FBS (Gibco) and 25 ug/ml gentamicin. Confluent flasks were treated with 0.25% trypsin and 1 mM EDTA, centrifuged, and resuspended in DMEM/F12. Cells were counted with a hemocytometer and seeded onto scaffolds at a density of 2 million cells/scaffold.

Cytokine Conditioning:

LF seeded foams were cultured for three weeks in 10% FBS media supplemented with either transforming growth factor beta 1 (TGF-β1) or hepatocyte growth factor (HGF) (Peprotech, Rocky Hill, N.J.) at concentrations of 4 ng/ml and 20 ng/ml, respectively (n=4/group). Supplementation at these concentrations has been shown to significantly influence matrix production by vocal fold fibroblasts (Luo, Y., et al., Effects of growth factors on extracellular matrix production by vocal fold fibroblasts in 3-dimensional culture. Tissue Eng, 2006. 12(12): p. 3365-74). Cultures maintained in non supplemented media served as controls. Control and cytokine supplemented media was changed every 2-3 days

Mechanical Testing

Sample properties were measured with the aid of a uni-axial tensile test. Hydrated (PBS, pH=7.4) samples (n=3) were placed into custom grips, and deformed at a constant strain rate of 10%/s until failure using a material testing system incorporating a 1 kg load cell with a resolution of 0.015 g. Custom control software was used to control deformation rate and record force/elongation data. Prior to testing, samples were imaged and initial dimensions were measured from the digital images. For each scaffold, engineering stress versus strain curves were generated from load, and elongation data. Elongation was determined from grip displacement. From each curve the tan modulus and ultimate strength was calculated. The tan modulus was calculated from a linear curve fit to the stress-strain curve. The ultimate strength was calculated as the maximum engineering stress attained by the sample prior to failure.

Material Extraction and Serial Digestion

Following mechanical testing samples were rinsed in DI, frozen to −80° C. and lyophilized. Lyophilized samples were weighed and then soaked in the solvent DMAC for 72 hours at 37° C. to remove the PU foam. The extracted material was rinsed three times in DI and lyophilized. Yield was calculated relative to initial PU foam weight.

$$Yield = \frac{D M A C Extracted Material Weight ( mg )}{P U Foam Weight ( g )}$$

To evaluate material composition a subset of DMAC extracted samples (n=3) were serially digested beginning with 1 unit/ml chonrointinase ABC (Acorda Therapeutics, Hawthorne, N.Y.), followed by 0.5% collagenase IV (Worthington, Lakewood, N.J.), and finishing with a 0.1% sodium dodecyl sulphate (SDS), and 0.2 mg/ml DNAse solution. Samples were soaked in the respective digestion solution for 24 hours at 37° C. with agitation, rinsed in DI, lyophilized, and weighed between steps.

Collagen and GAG Quantification:

Collagen content was estimated from hydroxy-proline concentration. Hydroxy-proline concentration was determined from extracted samples (n=4/group) using a published technique (Edwards, C. A. and W. D. O'Brien, Jr., Modified assay for determination of hydroxyproline in a tissue hydrolyzate. Clin Chim Acta, 1980. 104(2): p. 161-7). Briefly, extracted samples were digested in a 6N HCL solution (4 hrs at 110° C.) and then neutralized with sodium hydroxide. Digested samples were mixed with a chloromine T solution (1:2) and incubated at room temperature for 20 minutes. A dimethyl-aminobenzaldehyde assay solution was added (1:2) and the mixture was incubated at 60° C. for 15 minutes. During this time a red chromophore develops. Chormophore intensity indicates h-proline concentration. Sample absorbance was read at 550 nm using a microplate reader. Cytokine conditioned values were compared against controls, and relative collagen yields were calculated.

Total sulphated GAG yield was measured using a dimethylene blue assay according to a published method (Farndale, R. W., C. A. Sayers, and A. J. Barrett, A direct spectrophotometric microassay for sulfated glycosaminoglycans in cartilage cultures. Connect Tissue Res, 1982. 9(4): p. 247-8). The assay is based on the shift in absorption maximum which occurs when the dye is complexed with sulfated glycosaminoglycans. Extracted samples (n=4/group) were dissolved in a digestion solution containing papain diluted (50 ug/ml) in a sodium acetate (50 mM) buffer with EDTA (2 mm) and dithioreitol (2 mM). The digestion solution was added to lyophilized samples in a ratio of 100 ul of solution for every 1-2 mg of sample. Samples were incubated overnight at 60° C. to promote digestion. Ten ul of digested sample solution was added to 100 ul of dimethylene blue assay solution and absorbance was read at 530 nm using a microplate reader. Sulphated GAG yields were calculated relative to controls.

Imaging

Samples were resin imbedded (Technovit 8100, Heraeus, Germany), thin sectioned (5 um) with the aid of a microtome (Jung R M 2035, Leica, Germany), and mounted on microscopic slides. Sections were stained with hemotoxylin and eosin (H&E) to visualize sample architecture.

Data Analysis:

The affect of cytokine conditioning on each of the measures (material properties, matrix accumulation, and matrix composition) was evaluated with a one way, three factor (control, TGF-β1, HGF) analysis of variance (ANOVA) test. A standard 0.05 level of significance was used. Post hoc comparisons were performed using Tukey's test. Differences in material structure were qualitatively compared between samples from histological images.

Results

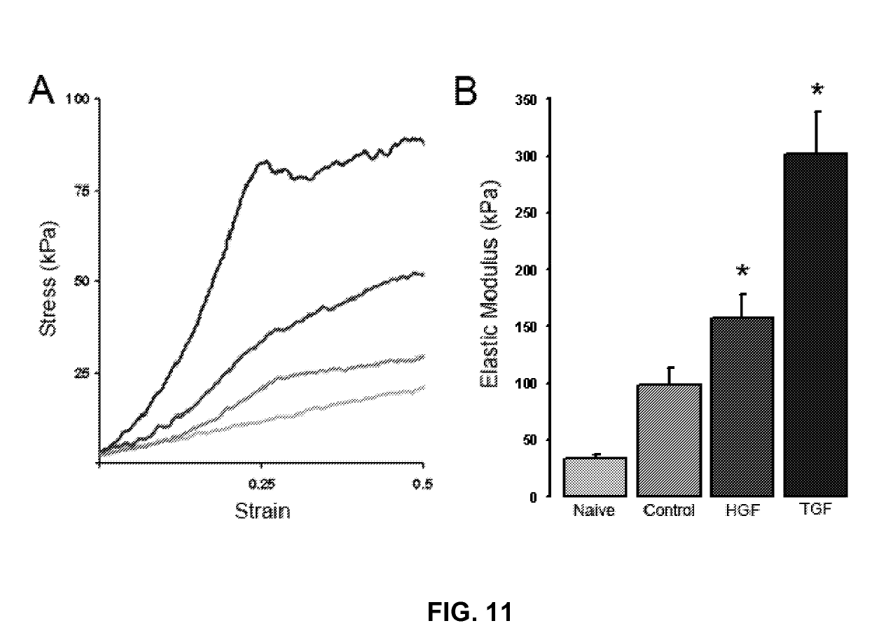

Stress-strain curves were generated for control, TGF-β1, and HGF conditioned samples. All samples displayed an initial linear loading region followed by a clear yield point at approximately 25% strain (FIG. 11). To quantify construct elastic modulus, a linear regression was used to fit the region of the stress strain curve extending from 5% strain to the yield point. Calculation of the elastic modulus for the pre-yield region indicates that both HGF and TGF-β1 conditioning resulted in statistically significant modulus increases when compared to static controls. Three weeks of culturing in HGF spiked media increased the modulus by 59%, from 99±14 kPa for controls to 157±21 kPa. TGF-β1 stimulated increase was more pronounced. TGF-β1 stimulated elastic modulus increased by 204% to 301±37 kPa. The average TGF-β1 modulus was significantly increased over HGF stimulated values.

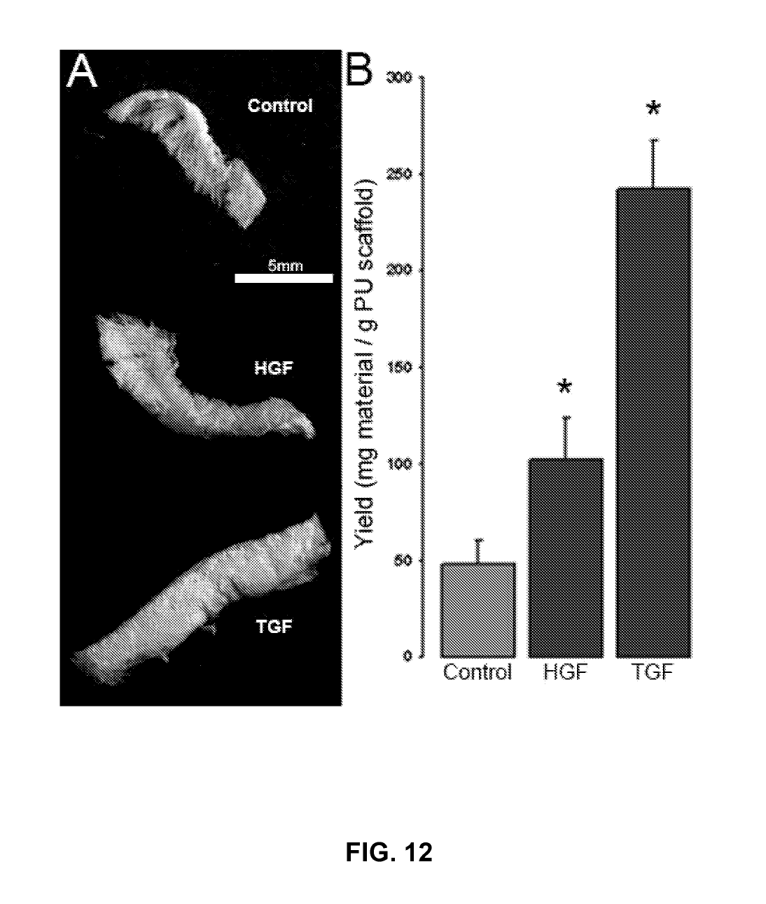

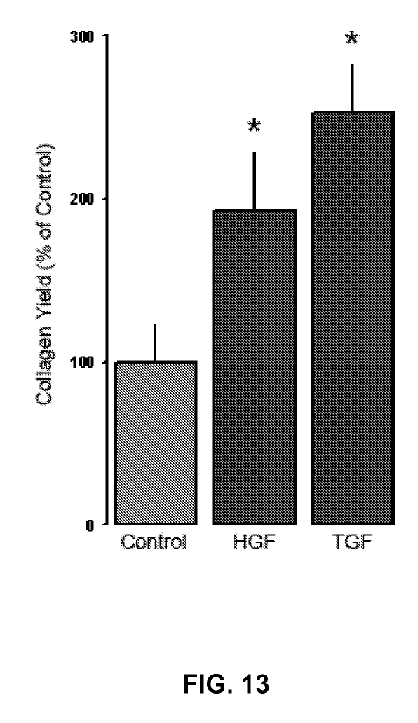

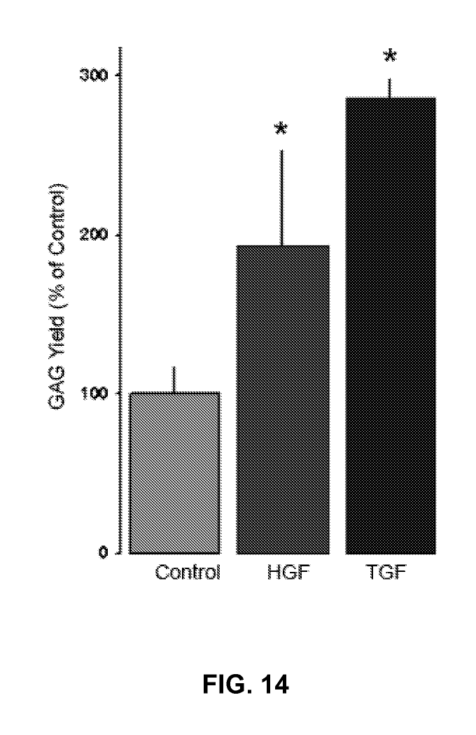

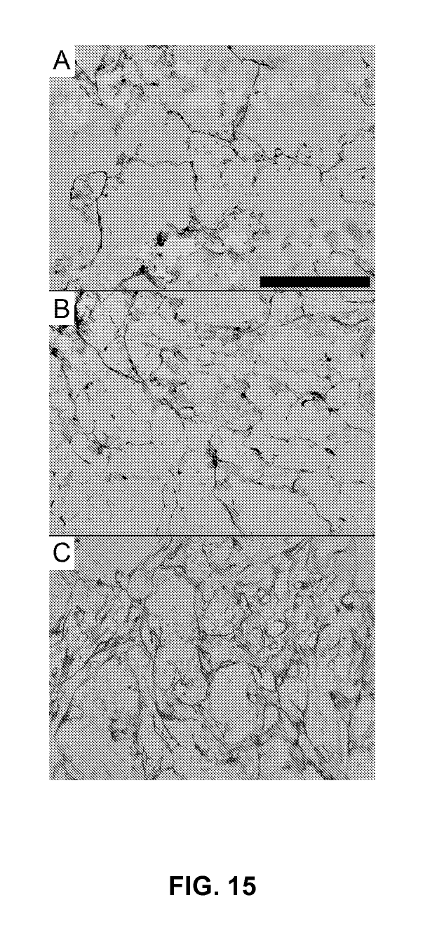

Material yield values were significantly increased for samples conditioned in either HGF or TGF-β1 spiked media (FIG. 12). Control scaffolds yielded 48±12 mg of material for every gram of PU scaffold seeded. Yield values increased to 102±21 mg and 243±25 mg for HGF and TGF-β1 treated samples. TGF-β1 conditioning resulted in a nearly five fold increase in material yield when compared to controls. Average collagen yield was significantly increased for both HGF and TGF-β1 conditioned samples. Collagen yield values for HGF and TGF-β1 treated samples were 195±35% and 255±25% of control values respectively (FIG. 13). Similarly, sulphated GAG yield was significantly increased for both HGF and TGF-β1 conditioned samples by 193±60% and 286±11% over control values respectively (FIG. 14). Microscopic examination of H&E stained thin sections revealed that TGF-β1 stimulated material was organized into a network that was notably denser than control material, with HGF stimulated network density intermediate to TGF-β1 and controls (FIG. 15).

Serial digestion of control samples resulted in complete degradation of the DMAC extracted material. Chondroitinase ABC and collagenase treatment removed 10% and 75% of the extracted material respectively. The remaining 15% was fully digested following SDS and DNAse treatment. Cytokine spiking resulted in small but statistically significant changes to the degradation profiles for HGF and TGF-β1 conditioned samples (Table 1). Chondroitinase treatment degraded 11% and 9% of HGF and TGF-β1 conditioned materials respectively, while collagenase treatment degraded 69% and 79% respectively. Although degradation differences were not significant between cytokine conditioned and controls they were significantly different from each other (HGF vs. TGF-β1).

TABLE 1
Table 1. Serial digestion of control, HGF and TGF-β1 conditioned
samples revealed modest but statistically significant shifts in
degradation profiles (mean ± SEM, n = 4).

| | | Treatment | | |
|---|---|---|---|---|
| | | Control | HG | TG |
| % Mass Digested | Chondroitinase | 9.7 ± 0.9 | 11.0 ± 0.5 | 9.1 ± 0.4 |
| | Collagenase | 73.6 ± 2.5 | 69.0 ± 4.1 | 78.5 ± 0.9 |
| | SDS and DNAse | 15.7 ± 3.8 | 18.5 ± 5.3 | 12.2 ± 0.4 |

The finding of this set of experiments can be summarized as follows. HGF and TGF-β1 treatment significantly increased both material yield and elastic modulus. TGF-β1 conditioning was a stronger promoter of yield and elastic modulus increases than HGF. HGF and TGF-β1 yield increases were in part due to increased collagen and GAG yield. Cytokine stimuli were capable of producing small but significant shifts in material composition. TGF-β1 conditioning samples have elevated collagen concentrations while HGF samples have elevated GAG concentration. Aside from increases in material density, cytokine stimulation is not capable of influencing the organization of material architecture.

### Examples 3

### Effects of Mechanical stimuli on the 3-D ECM

Methods

Substrate Preparation:

Polyurethane pellets (4 g) (Tecoflex SG-8, Thermedics) were dissolved in dimethylacetamide (40 ml) (DMAC) overnight at 60° C. Pluronic 10R5 (20 ml) (BASF) was added and the solution thoroughly mixed. The polymer solution is cooled to 46° C., quenched in a ethanol/dry ice bath for 2.5 minutes, and precipitated overnight in DI water. Substrates are separated from the molds, rinsed in DI water for 48 hours, frozen to -80° C., and lyophilized. Lyophilized scaffolds are sectioned (30mm×10 mm×2mm) and attached to Mylar mounts using medical grade UV curable adhesive (MD 1180-M, Dymax, Torrington, Conn.). Prepared scaffolds were ethylene oxide sterilized at the University of Utah Hospital sterilization facility. To facilitate cellular attachment, scaffolds were soaked overnight in a fibronectin solution (20 ug/ml) (Invitrogen).

Cell Culture:

For all treatment and control conditions, human laryngeal fibroblasts (LFs) were used. Cells were originally isolated from a sample of human tracheal tissue obtained by biopsy from a patient undergoing surgery. Frozen larygenal fibroblasts were thawed, plated in T-175 flasks, and grown to confluency in DMEM/F12 (Gibco) with 10% FBS (Gibco) and 25 ug/ml gentamicin (Sigma). Confluent flasks were disassociated with 0.25% trypsin and 1mM EDTA, centrifuged, and resuspended in DMEM/F12. Cells were counted with the aid of a hemocytometer and seeded onto scaffolds at a density of 2 million cells/ scaffold.

Mechanical Stimulation

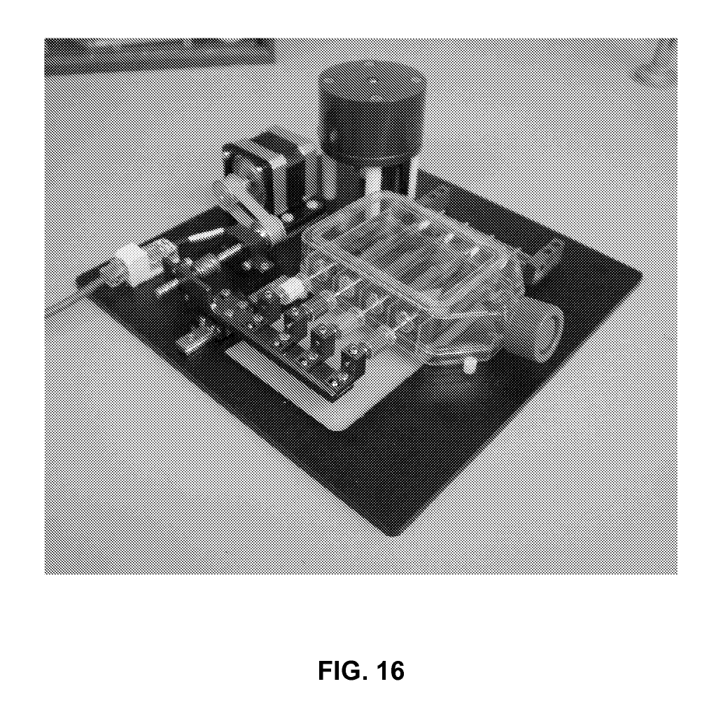

To provide mechanical stimulation a custom designed vocal fold bioreactor was used (FIG. 16). Bioreactor fabrication, characterization, and function were discussed in previous publications (Titze, I. R., et al., Design and validation of a bioreactor for engineering vocal fold tissues under combined tensile and vibrational stresses. J Biomech, 2004. 37(10): p. 1521-9 and Titze, I. R., et al., Strain distribution in an elastic substrate vibrated in a bioreactor for vocal fold tissue engineering. J Biomech, 2005. 38(12): p. 2406-14). Three sample groups were conditioned eight hours a day for three weeks using the following stimuli (4 samples/group)
1. Static
2. Cyclically axial strain (10%, 0.25 Hz)
3. Dual mode strained plus vibration (100 hz)

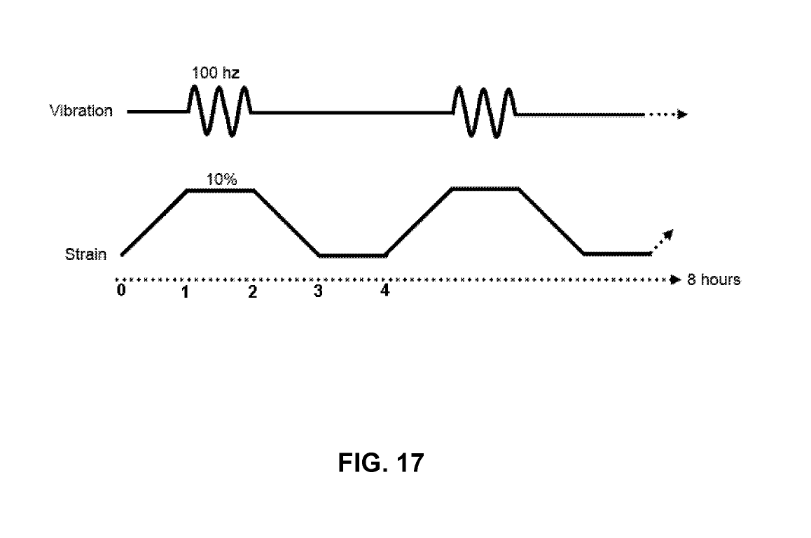

These values are within the physiological ranges for vocal strain, frequency, and duration (Fitch, J. L. and A. Holbrook, Modal fundamental frequency of young adults. Archives of Otolaryngology, 1970. 92: p. 379-382 and Titze, I. R., J. J. Jiang, and E. Lin, The dynamics of length change in canine vocal folds. J Voice, 1997. 11(3): p. 267-76). For dual mode conditioning, peak axial strains are synchronized to the vibration stimulation (FIG. 17). The vibration and strain conditioning protocol was as follows:

Vibration: Each cycle (0.25 Hz) consisted of one second of vibration (100 hz) followed by three seconds of rest. Total vibration sums to 2 hours over the 8 hour conditioning period, a duration consistent with heavy vocal usage.

Strain: Each cycle (0.25 Hz) consisted of one second at 10% strain proceeded and followed by 1 second of increasing and decreasing strain (*105/sec) all followed by one second of rest.

For dual mode treatment, vibration and strain stimuli were applied in combination, with stain and vibration cycles synchronized such that the 1 second of vibration coincided with the 1 second of maximum strain. The chosen pattern approximates the vocal straining and vibration during speech. Growth medium containing DMEM F12 supplemented with 10% FBS, gentamicin, and 1 mM ascorbic acid was changed every 2-3 days.

Mechanical Testing

Sample properties were measured with the aid of a uni-axial tensile test. Hydrated (PBS, pH=7.4) samples (n=4) were placed into custom grips, and deformed at a constant strain rate of 10%/s until failure using a material testing system incorporating a 1 kg load cell with a resolution of 0.015 g. Custom control software was used to control deformation rate and record force/elongation data. Prior to testing, samples were imaged and initial dimensions were measured from the digital images. For each scaffold, engineering stress versus strain curves were generated from load, and elongation data. Elongation was determined from grip displacement. From each curve the tan modulus and ultimate strength was calculated. The tan modulus was calculated from a linear curve fit to the stress-strain curve. The ultimate strength was calculated as the maximum engineering stress attained by the sample prior to failure.

Material Extraction

Following mechanical testing samples were rinsed in DI, frozen to −80° C. and lyophilized. Lyophilized samples (n=4) were weighed and then soaked in the solvent DMAC for 72 hours at 37° C. to remove the PU foam. The extracted material was rinsed three times in DI and lyophilized. Yield was calculated relative to initial PU foam weight.

$$Yield = \frac{D M A C Extracted Material Weight ( mg )}{P U Foam Weight ( g )}$$

GAG Quantification:

Total sulphated GAG yield was measured using a dimethylene blue assay according to a published method (Farndale, R. W., C. A. Sayers, and A. J. Barrett, A direct spectrophotometric microassay for sulfated glycosaminoglycans in cartilage cultures. Connect Tissue Res, 1982. 9(4): p. 247-8). The assay is based on the shift in absorption maximum which occurs when the dye is complexed with sulfated glycosaminoglycans. Extracted samples (n=4/group) were dissolved in a digestion solution containing papain diluted (50 ug/ml) in a sodium acetate (50 mM) buffer with EDTA (2 mm) and dithioreitol (2 mM). The digestion solution was added to lyophilized samples in a ratio of 100 ul of solution for every 1-2 mg of sample. Samples were incubated overnight at 60° C. to promote digestion. Ten ul of digested sample solution was added to 100 ul of dimethylene blue assay solution and absorbance was read at 530 nm using a microplate reader. Sulphated GAG yields were calculated relative to controls.

Imaging

Samples were resin (Technovit 8100, Heraeus-Kulzer, Germany) embedded, thin sectioned (5 um) with the aid of a microtome (Jung R M 2035, Leica, Germany), and mounted on microscopic slides. Sections were stained with hemotoxylin and eosin (H&E) to visualize sample architecture.

Data Analysis:

The affect of mechanical stimuli on material properties, matrix accumulation, and GAG concentration was evaluated with a one way, three factor (control, axial, axial+vibration) analysis of variance (ANOVA) test. A standard 0.05 level of significance was used. Post hoc comparisons were performed using Tukey's test. Differences in material structure were qualitatively compared between samples.

Results

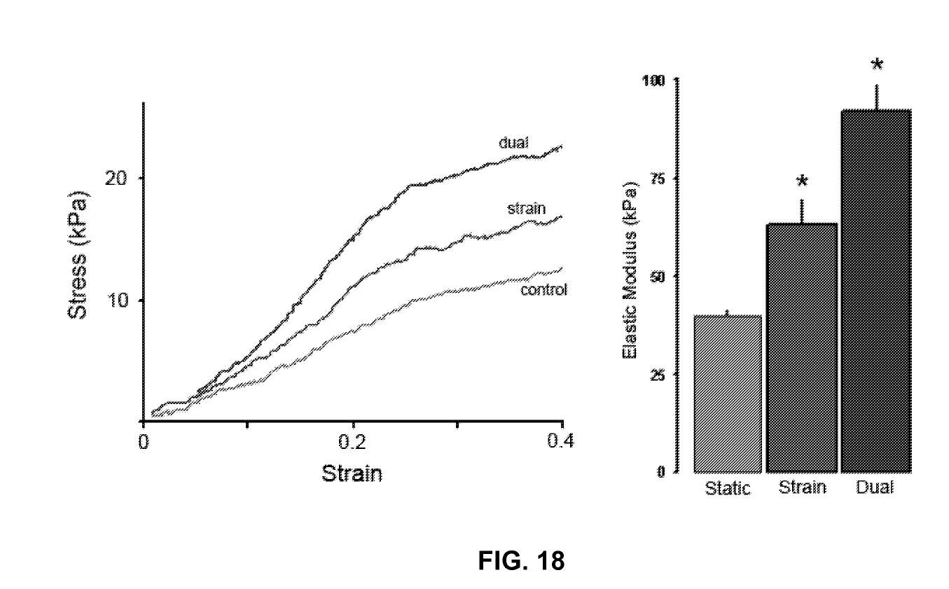

Stress-strain curves were generated for control, strain, and dual mode conditioned samples. Consistent with previous results, stress-strain curves for control and mechanically conditioned samples were characterized by a linear region, a yield point at around 25% strain, and a second linear region (FIG. 18). To quantify construct elastic modulus, a linear regression was used to fit the region of the stress strain curve extending from 5% strain to the yield point. Both strain and dual mode conditioning significantly increased construct stiffness. Static control stiffness increased from 40±2 kPa to 63±7 kPa following strain conditioning and 92±7 KPa following dual mode conditioning. Conditioning did not affect the location of the yield point.

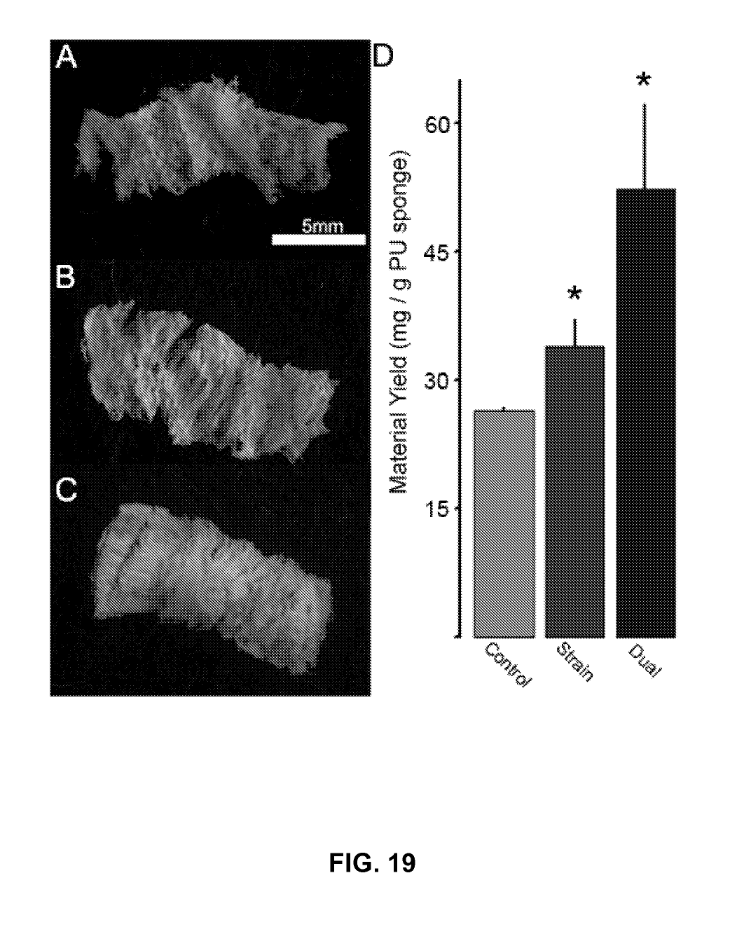

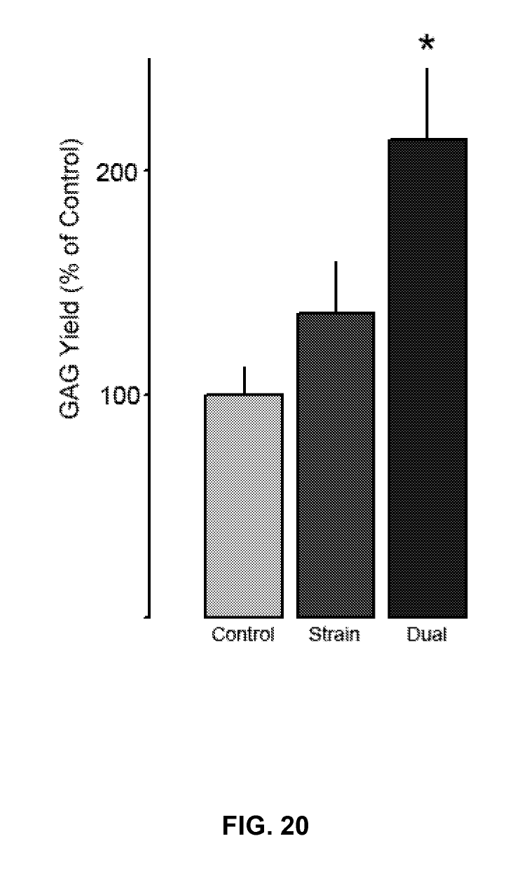

Material was collected from all samples following DMAC extraction. Material yield for both axial and dual mode conditioning samples was significantly increased over static controls (FIG. 19). Static control samples yielded 25±1 mg of material for every gram of PU foam seeded. Following strain and dual mode conditioning yield increased to 34±3 mg and 52±10 mg respectively. Relative GAG yield was significantly increased for both strain and dual mode conditioned samples (FIG. 20). Strain conditioned yield increased by 37±23% while dual mode conditioning increased yield by 114±32%. Dual mode conditioned GAG yield was also significantly increased over strain conditioned values.

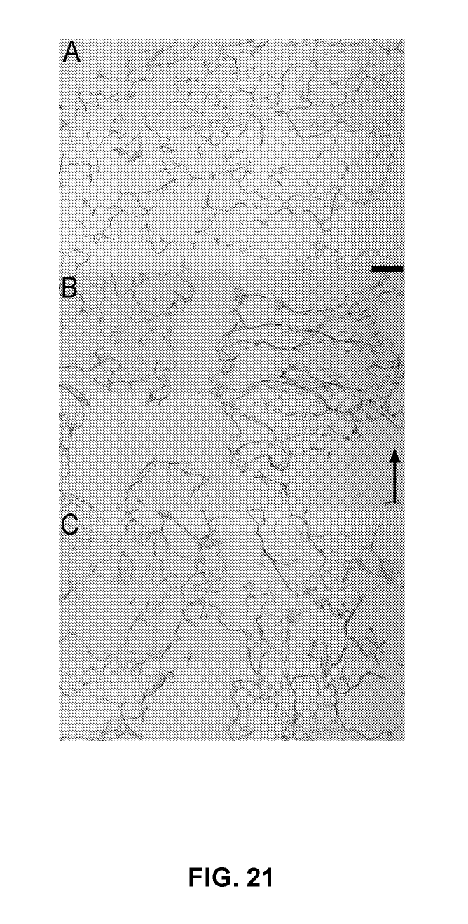

Microscopic examination of H & E stained thin sections revealed a porous network of cell remnants and ECM for both control and conditioned samples (FIG. 21). While network porosity was not visibly affected by mechanical conditioning, the cell and ECM network segments for strain and dual mode conditioned samples appear thicker than the fine network observed for control samples. H&E stained samples were also visually evaluated for evidence of network organization. Network alignment was not apparent for either control or dual mode conditioned samples. However, strain conditioned sample network segments were noticeably aligned in the direction running perpendicular to the applied axial strain.

The findings of this example can be summarized as follows. The GAG production, yield, and elastic modulus for dual mode conditioned samples was double that of static controls. The addition of high frequency vibration to axial strain conditioning increased GAG production, material yield, and sample elastic modulus by approximately 50%. Strain induced material alignment was eliminated with the addition of vibration stimulation. The mechanism responsible for vibration sensitivity are unclear, however strain, fluid flow, and inertial forces induced by vibration are likely causes.

### Example 4

### Effect of Bioreactor Induced Vibrational Stimulation on ECM

Methods:

Vibrational Bioreactor

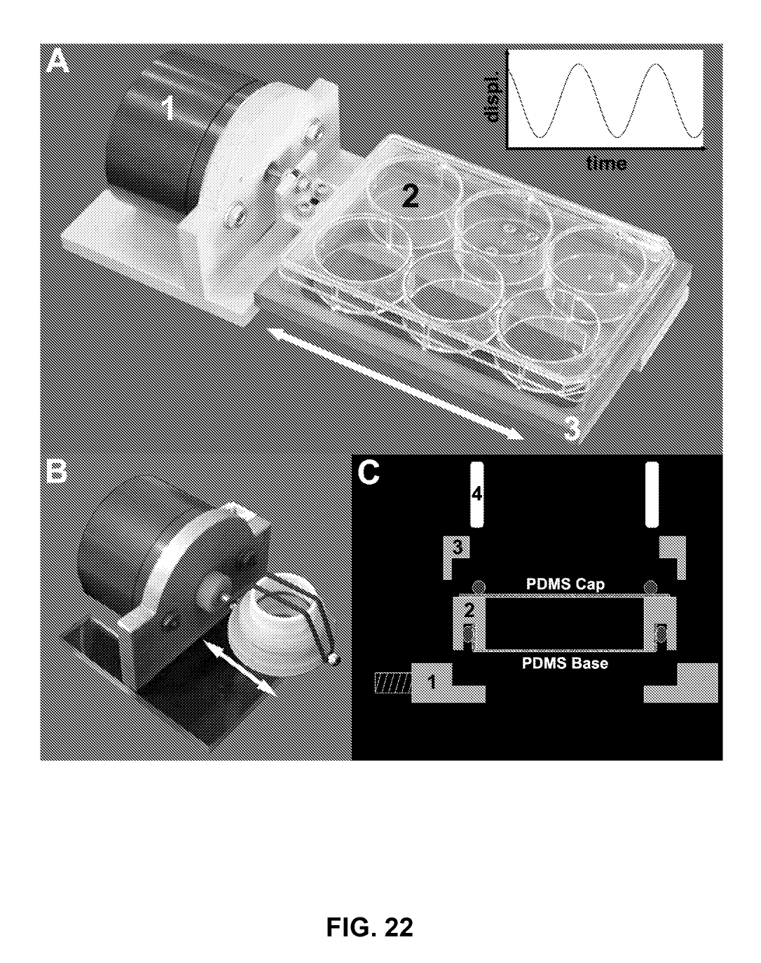

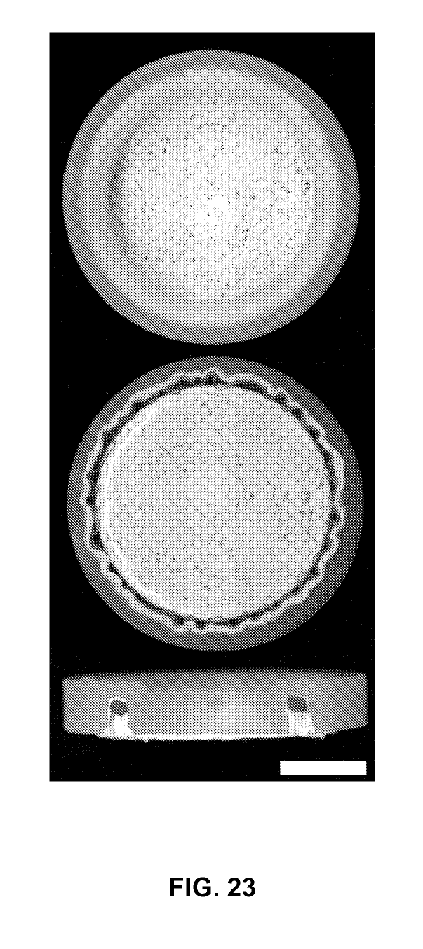

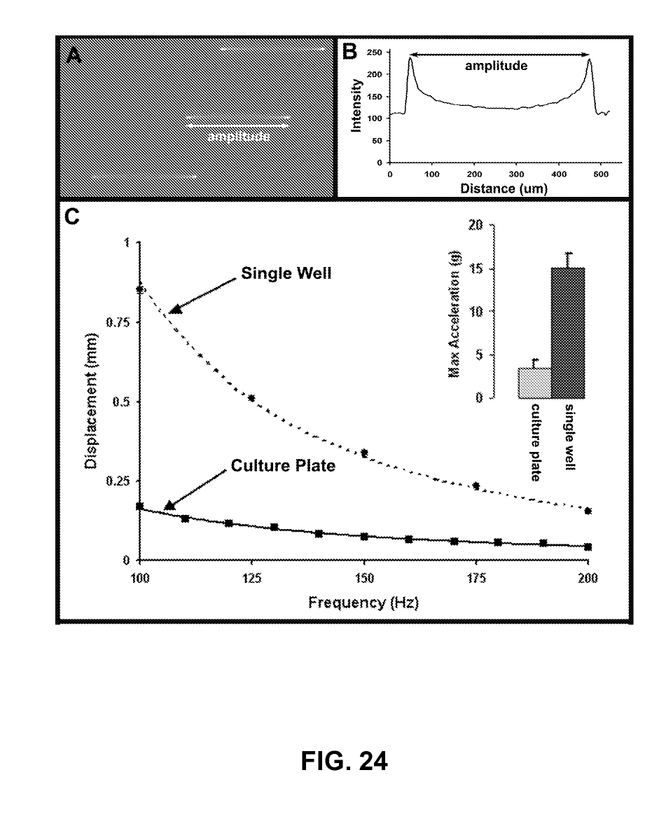

To study the affect of high frequency mechanical loading on cell behavior four vibratory bioreactors were designed, fabricated, and characterized. The vibration is driven by an electromagnetic voice coil actuator (BEI Kimco, Vista, Calif.) which is powered by a sinusoidal voltage signal supplied and controlled by an analog output board and control software (Labview, National Instruments, Austin, Tex.) running on a PC. The software allows the user to control vibrational frequency, amplitude and duty cycle. The voice coil actuator is attached to a polymer base which can accommodate a standard tissue culture plate (FIG. 22), or following a change in bases the actuator can also drive a custom fabricated culture well (dia.=25 mm). To form the bottom of the well, either a planar PDMS membrane, or an open celled polyurethane foam sheet is press fit into place (FIG. 23). To measure the vibrational intensity for both mount types, fluorescent microspheres (Molecular Probes, Oreg.) were adsorbed to the bottom of both a single well (with PDMS base) and 6 well culture plate. When vibrated and viewed microscopically the spheres are seen as bright streaks which trace the vibrational path (FIG. 24). The traces were imaged across a range of frequencies and a multiple surface points for both the culture plate and single well configurations. Vibration amplitudes were measured directly from the traces using image analysis software (ImagePro, Media Cybernetics, MD). For each trace, the maximum acceleration was calculated as the second derivative assuming a sinusoidal displacement pattern with time.

Polyurethane Foam Preparation

Polyurethane pellets (4 g) (Tecoflex SG-8, Thermedics) were dissolved in dimethylacetamide (40 ml) (DMAC) overnight at 60° C. Pluronic 10R5 (20 ml) (BASF) was added and the solution thoroughly mixed. The polymer solution was cooled to 46° C., pored into polymer molds, cooled for 2.5 minutes in a dry-ice/ethanol bath, and precipitated overnight in a DI water bath. The resulting foam sheets have an interconnected pore network and high flexibility. Sheets (40×40×0.5 mm) were separated from the molds, rinsed in DI water for 48 hours, frozen to −80° C., and lyophilized. Lyophilized sheets were ethylene oxide sterilized at the University of Utah Hospital sterilization facility.

Experimental Set-up

The experiments were designed to assess cellular responses that could influence tissue assembly. Specific experiments included 1) assessment of matrix and matrix related gene expression using a DNA microarray 2) measurement of soluble cytokine levels in conditioned media using enzyme linked immuno-sorbent assays (ELISA), 3) evaluation of matrix protein deposition on conditioned samples using immuno-fluorescent labeling, and 4) measurement of conditioned sample material properties using a uni-axial tensile test.

For all experiments, frozen laryngeal fibroblasts originally obtained during patient biopsy were thawed, plated in T-175 flasks, and grown to confluency in DMEM/F12 with 10% FBS and 25 ug/ml gentamicin. Confluent flasks were disassociated with 0.25% trypsin and 1mM EDTA in HBSS, centrifuged, and resuspended in serum free DMEM/F12. Depending on the assay, cells were planted onto either 6 well plates (DNA microarray), planar PDMS membranes (cytokine levels), or porous polyurethane scaffolds (matrix accumulation and mechanical testing). To facilitate cell attachment to the PDMS membranes and polyurethane scaffolds each was immersed overnight in a solution of 20 ug/ml bovine fibronectin in phosphate buffered saline (PBS), and rinsed with sterile distilled water (DI). Once plated, cells were maintained in serum free Sato-media (Sato, J. D. and M. Kan, Media for culture of mammalian cells. Curr Protoc Cell Biol, 2001. Chapter 1: p. Unit 1 2) for 48 hours prior to conditioning. Six well and PDMS planar substrates had each formed confluent layers prior to the initiation of testing. Similarly, cells were densely distributed throughout the porous scaffolds. Prior to conditioning, serum free media was changed to DMEM/F12 supplemented with 1 mM ascorbic acid (Sigma) and 10% FBS.

Gene Expression Analysis

With a bioreactor configured for culture plates, a cell seeded six well plate was vibrated at 100 Hz, a frequency that falls within the male speaking range (Fitch, J. L. and A. Holbrook, Modal vocal fundamental frequency of young adults. Arch Otolaryngol, 1970. 92(4): p. 379-82), for 14 minutes distributed over a 6 hour conditioning period. The conditioning pattern consisted of 1.5 seconds of vibration followed by a 30 second static period, repeated for six hours. Six hour conditioning periods were repeated once a day for 3 days. Static cell cultures were maintained as controls. At the completion of the experiment, total RNA was isolated from the vibrated and static control wells using Trizol reagent. Sample mRNA was amplified into aRNA using RiboAmp (Molecular Devices, Sunnyvale, Calif.) amplification process. Cy3 and Cy5 labeled cDNA was reverse transcribed from vibrated and control sample aRNA, respectively by the University of Utah DNA microarray facility. Labeled cDNA was hybridized to a microarray slide containing 9600 human gene clones in duplicate. Fluorescence ratios (green:red) were used to quantify differences in gene expression between the vibrated and non-vibrated controls. The mean expression ratio and standard deviation was calculated for the population of genes tested. Matrix and matrix related genes of interest with expression ratios two standard deviations above the mean were identified.

Measurement of Media Cytokine Levels

With the bioreactors converted to accommodate the single well, samples with PDMS membrane bases were vibrated (100 Hz) for 2 hours distributed over a 6 hour conditioning period (one second of vibration followed by a two second static period). After twenty four hours, media from vibrated and static control cultures (N=4 samples/condition) was collected. Following collection, cells were fixed, permeabilized, and cell nuclei were stained with DAPI. Fifteen macroscopic fields (200×) were obtained and used to estimate cell density and total cell number for each culture.

Media levels for the profibrotic cytokine transforming growth factor beta 1 (TGF-β1) was determined using a sandwich ELISA. Ninety six well high protein binding ELISA plates (Greiner Bio-one, Germany) were coated with 100 ul of lug/ml anti-TGF-β1 capture antibody (R&D Systems, MN) overnight at room temperature. Plates were washed three times with tris buffered saline containing 0.05% Tween 20 (TBST) and blocked with a 1% BSA solution for one hour at room temperature. In all samples, latent TGF-β1 was activated through an acidification step involving a 1:5 mix of 1N HCL and sample followed by a ten minute incubation. Samples were neutralized after incubation using a solution of 1.2N NaOH and 0.5M Hepes added 1:4.2 to acidified samples. One hundred microliter aliquots of TGF-β1 standards (Peprotech, State) or activated samples were added to wells and incubated for two hours at room temperature. After washing, 100 ul of 0.1 ug/ml biotin labeled anti-TGF-β1 detection antibody (R&D Systems, MN) was added to wells and incubated at room temperature for two hours. Plates were again washed and 100 ul of 0.2 ug/ml peroxidase conjugated steptavidin was added to each well, incubated at room temperature for 30 minutes, and washed. Following the addition of 100 ul aliquots of fluorogenic substrate absorbance was kinetically read (16 readings over 20 minutes) at 390 nm by a microplate reader. Sample concentrations were extrapolated from the standard curve.

MCP-1 concentration was determined using a sandwich ELISA process similar to that reported for TGF-⊕1. Mouse anti MCP-1 monoclonal antibody and biotintylated goat anti-MCP1 polyclonal antibody (R&D Systems, MN) were used as capture (2 ug/ml) and detection (0.2 ug/ml) antibodies respectively. Recombinant human MCP-1 (Peprotech, N.J.) was used to generate a standard curve.

Measurement of Matrix Accumulation

With the bioreactors in the single well configuration, samples containing cell seeded porous polyurethane foam bases were vibrated (100 Hz) for 2 hours distributed over a 6 hour conditioning period. This cycle was repeated once a day for either three days (short term) or twenty-one days (long term) (N=4/time point). Media was changed every 1-2 days throughout the conditioning period. Following the short three day conditioning period, vibrated and static control samples were fixed in 4% paraformaldehyde, permeabilized with 0.05% triton and stained for the presence of collagen type I (Sigma 500:1) and insoluble cellular fibronectin (400:1) followed by the appropriate fluorescently labeled secondaries. The cellular fibronectin antibodies selected do not cross react with the bovine fibronectin used to coat the PDMS and polyurethane. The immunoreactivity to collagen and fibronectin for each sample was used to estimate protein accumulation. Immunoreactivity was quantified from fluorescence intensity for each scaffold. Florescence intensity for conditioned and control samples was measured with the aid of a mircoplate reader (Synergy HT, Bio-Tek Instrument Inc., VT). Cells were counterstained with the nuclear die DAPI. Fluorescence values were normalized to cell count for each sample. Cell counts were estimated from DAPI images. Confocal microscope images were also obtained for qualitative comparison.

Mechanical Testing

Matrix accumulation within the long term 21 day conditioned samples was assessed through mechanical testing followed by extraction of the accumulated material from the polyurethane foam base. Long term vibration conditioned and static control sample material properties were evaluated with a uni-axial tensile test (N=3 samples/group). Hydrated (PBS, pH=7.4) samples were sectioned into strips (20 mm×19 mm×0.5 mm), placed into grips, preloaded (5 g) to remove any laxity, and then deformed at a constant strain rate of 10%/s using a material testing system (Tol-o-matic, Hamel Minn.) incorporating a 1 kg load cell. Prior to testing the load cell was calibrated using a series of weight standards ranging from 20 to 750 g producing a linear curve relating output voltage to load. At the loads measured (<200 g) load train displacement was negligible and therefore sample strain was measured directly form crosshead displacement. Custom control software (Labview) was used to control deformation rate and record load/elongation data.

Following mechanical testing long term vibrated and control samples were fixed in 4% paraformalehyde and soaked in the solvent dimethylacrilamide (DMAC) for 24 hours at 37° C. to remove the polymer scaffold. The extracted material was rinsed in DI, frozen and lyophilized. Lyophilized samples were photographed and then resin imbedded, thin sectioned (5 um), and stained with hemotoxylin and eosin to identify matrix architecture.

Statistics

A single sided students t-test was used to evaluate cytokine level, matrix accumulation, and scaffold stiffness differences between vibrated constructs and controls. All data is displayed as the mean and standard error of the mean (SEM).

Results

Bioreactor Characterization

The voice coil actuator and control software were capable of producing measurable vibrations across a range of frequencies (100-200 Hz) for both the culture plate and single well configurations (FIG. 24). Vibrational amplitudes were maximal (0.9 mm at 100 Hz) for the lighter single well configuration. The increased mass of the culture plate configuration reduced vibration amplitudes to approximately 20% of single well values. Measured amplitudes were seen to decrease with the inverse square of the frequency. The inverse relationship between displacement and frequency agrees with the theoretical model of the bioreactor as a mass and spring system. The maximum acceleration was stable across the range of frequencies measured, but did vary with mass. These findings agree with a spring and mass model. As a result of the increased mass, average maximum accelerations for the culture plate configuration was approximately 20% of culture well configuration values (FIG. 24 inset).

DNA Microarray Analysis

The expression of several matrix and matrix related genes were affected by high frequency vibration (Table 2). The types of genes that were affected by vibration could be broadly classified as either matrix protein genes (collagen I and IX, syndecan, laminin), inhibitors of matrix degradation genes (TIMP 1 and 3), or profibrotic cytokine genes (CTGF and PDGF).

TABLE 2
DNA microarray summary with ECM and ECM related genes of interest
with expression ratios.

| | Expression Ratio |
|---|---|
| Gene | (Vibrated:Static Control) |
| Tissue Inhibitor of Metalloprotease 1 | 4.2 |
| (TIMP1) | |
| Tissue Inhibitor of Metalloprotease 3 | 2.7 |
| (TIMP3) | |
| Collagen Type I | 2.5 |
| Lysyl Oxidase | 2.1 |
| Transforming Growth Factor Beta1 | 1.7 |
| Latent Binding Protein | |
| Syndecan | 1.6 |
| Laminin | 1.6 |
| Connective Tissue Growth Factor | 1.5 |
| Platelet Derived Growth Factor | 1.5 |
| Collagen Type IX | 1.5 |

Cytokine Media Levels

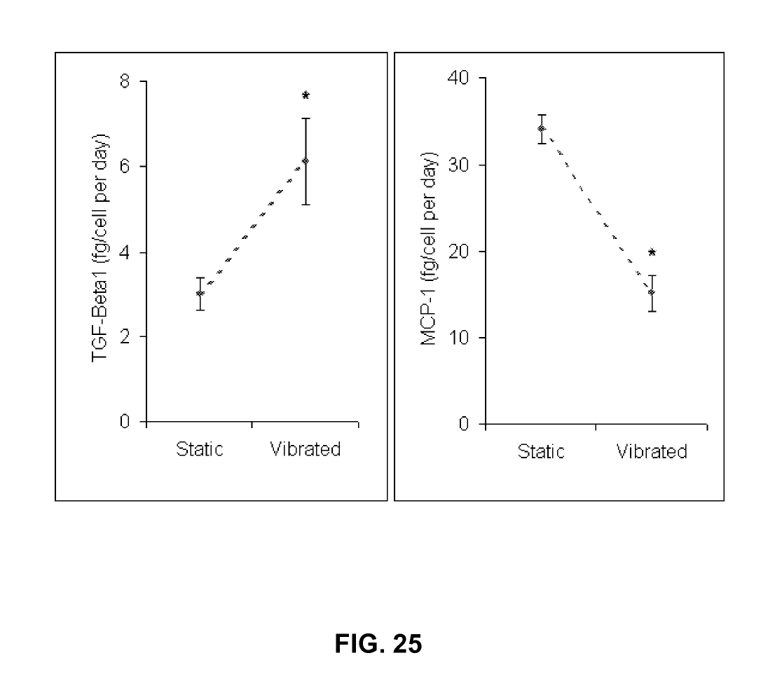

Both MCP-1 and TGF-β1 media levels were affected by high frequency vibration (FIG. 25). To account for variations in cell density, ELISA measured protein levels were normalized to cell counts for all conditioned and control cultures. Following vibration conditioning, TGF-β1 levels were significantly increased when compared to static controls (p<0.01). Conditioned TGF-β1 medial levels doubled from static levels of 3.0±0.6 fg/cell to 6.1±0.3 fg/cell. Conversely, conditioned MCP-1 media levels were significantly decreased when compared to controls (p<0.01). Vibration conditioned MCP-1 levels of 15±1.7 fg/cell were less than half that of control levels (34±2.2 fg/cell).

Matrix Immunostaining

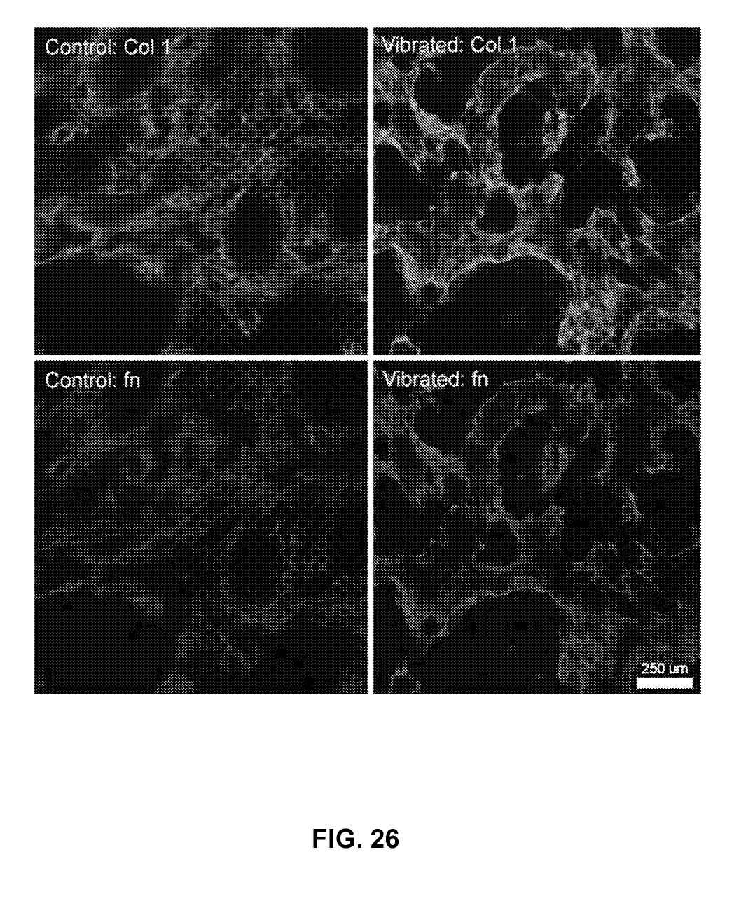

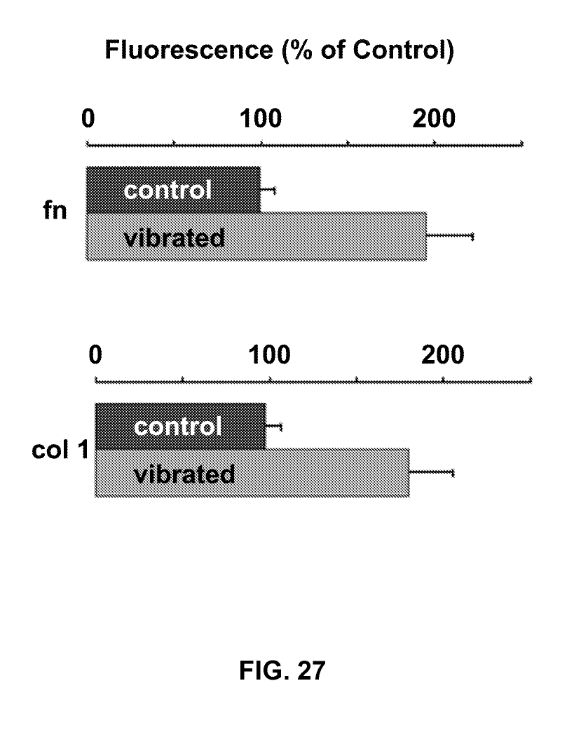

Using immunostaining, fibronectin and collagen type 1 accumulation as well as fibroblast distribution was evaluated following short term conditioning for both vibrated and control porous polyurethane samples. At the end of the three day conditioning period, laryngeal fibroblasts were well distributed throughout the scaffold for both control and vibrated cultures. Immuno-histological analysis, demonstrated that the immunoreactivity to both cellular fibronectin and collagen type I was both qualitatively and quantitatively increased when compared to unconditioned controls (p≦0.05) (FIGS. 26 and 27).

Mechanical Testing

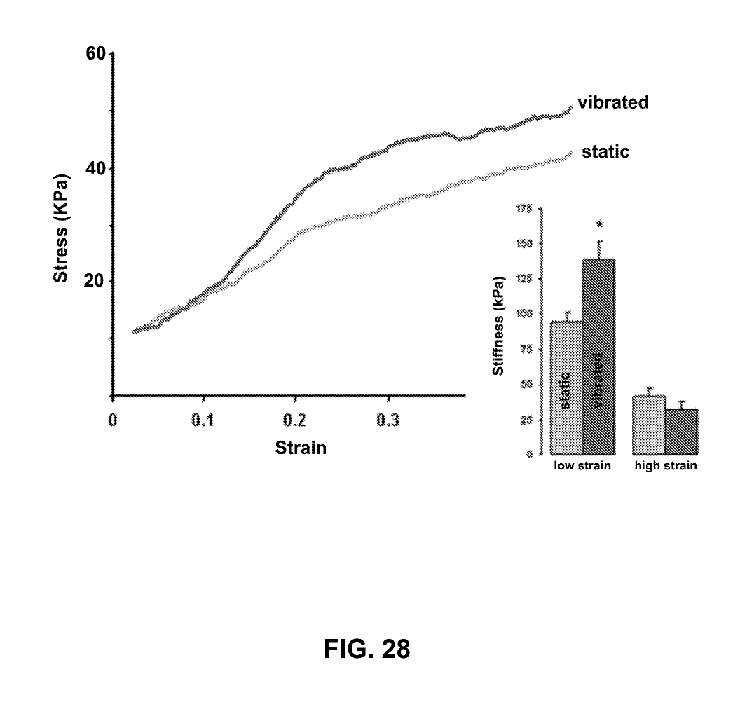

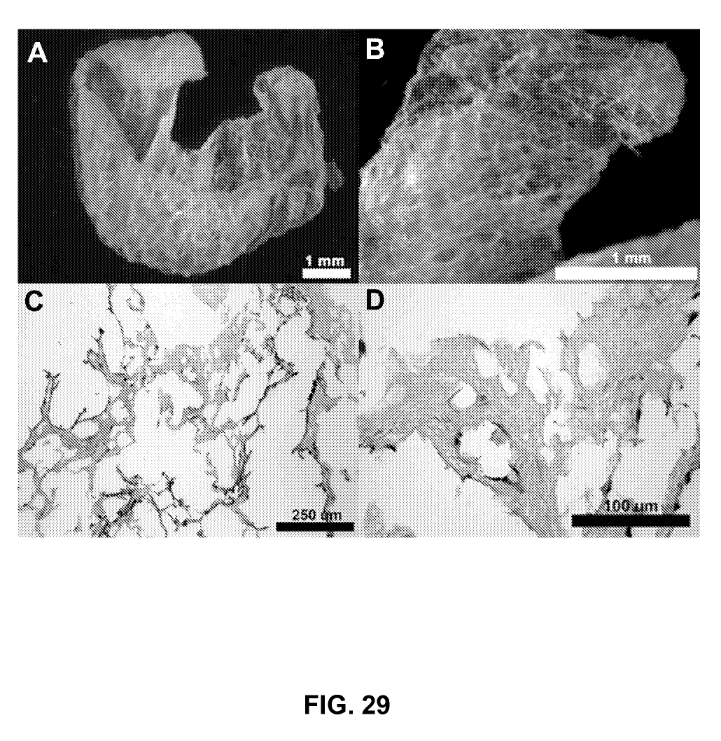

Stress-strain curves were generated for 21 day vibration conditioned and control samples. For all samples, stress-strain curves were characterized by a linear low strain region, a yield point at around 25% strain, and a second linear region which extended beyond 75% strain for all samples measured (FIG. 28). To quantify construct elastic modulus, separate linear regressions were used to fit both the low strain (5-25%) and high strain (30-75%) regions. In the low strain region, vibration conditioning significantly increased the modulus of the engineered construct when compared to static controls (FIG. 28 inset). Specifically, average sample modulus increased by 47% from 95±7 KPa to 130±13 KPa following vibration conditioning. Vibration did not affect the location of the yield point and in the high strain region elastic modulus was similar for both the vibrated and static samples. Accumulated material was successfully extracted from long term vibration conditioned and control samples (FIG. 29). H&E staining of samples revealed a porous yet continuous engineered material consisting of cells and accumulated matrix proteins. There was no evidence of residual polyurethane foam within the extracted material. Qualitative differences between material extracted from vibrated and control samples was not evident from the histological sections.

In this study we have shown that a vibrational bioreactor capable of mimicking the high frequency vibration patterns of the vocal cord can be fabricated and used to produce engineered matrix. In addition, several matrix and matrix related genes were upregulated in response to vibration conditioning. Collagen and fibronectin protein accumulation as well as media levels for the pro-fibrotic cytokine TGF-β1 were increased in response to vibration conditioning. The vibration conditioned samples were significantly stiffer than static controls.

### Example 5

### Engineering a Human Derived Extracellular Matrix Material Using Human Mesenchymal Stem Cells

Methods

Frozen human mesenchymal stem cell aliquots (ATCC, Manassas, Va.) were thawed, plated into culture flasks, and grown to confluency. Confluent cells were dissociated, counted with the aid of a hemocytometer, and seeded onto PU foams at a density of 2 million cells/foam. (the same density used for all laryngeal seeded samples) Samples were cultured for three weeks in a growth medium consisting of DMEM F12 supplemented with 10% FBS and 1 mM ascorbic acid. At the end of the three week conditioning period, samples (n=4) were tensile tested (10%/sec). Following tensile testing, the accumulated biological material was isolated from the PU foam using the previously described DMAC extraction process and yield was calculated.. Following extraction, a representative segment was resin embedded, thin sectioned, H&E stained, and imaged. Sample collagen content was determined using the previously described H-proline assay. To evaluate cellularity, a group of HMSC seeded samples were decellularized (0.1% SDS), and the remaining material was DMAC extracted

Results and Discussion

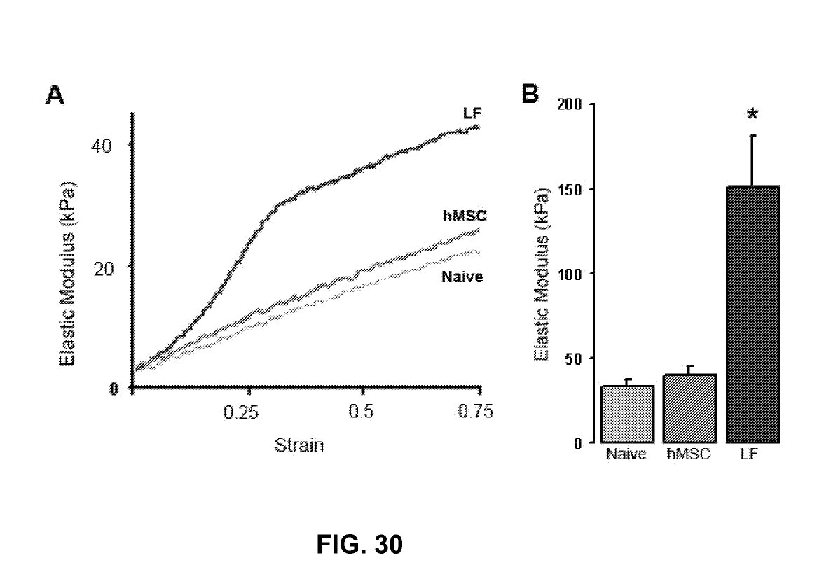

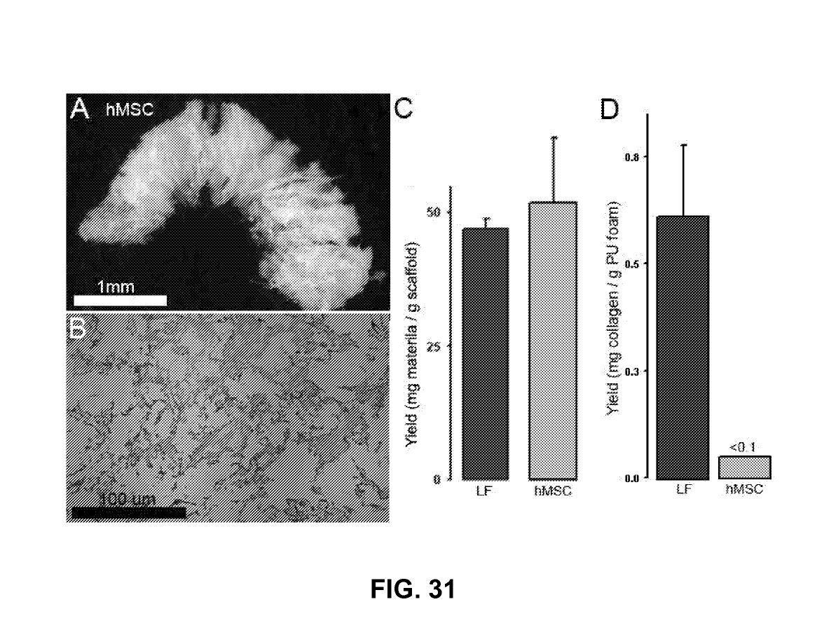

Despite the presence of viable and densely seeded hMSCs within the PU foam at the time of testing (calcein AM viability stain, data not shown), the elastic modulus was not significantly different from unseeded control scaffolds (FIG. 30). Notably, hMSC samples lacked the 25% strain yield point observed during testing of all previous LF seeded samples. The yield point likely indicates the mechanical failure of secreted extracellular matrix proteins under loading. The lack of a yield point suggests that hMSCs secreted little extracellular matrix during the three week conditioning period. However, the yield results indicate that the quantity of material extracted from hMSC samples is equivalent to LF seeded samples. Average material yield from hMSC seeded samples was 52 mg/g scaffold seeded (FIG. 31) versus 48 mg/g for LF seeded samples. Furthermore, the histology images indicate that the density and architecture of hMSC seeded samples is similar to LF samples (FIG. 31). Taken together, the tensile, yield, and histology results suggests that HMSC seeded samples, although similar in size and shape is formed by constituents that do not resist tensile loads. The decellularization and h-proline results indicate that the material extracted from hMSC samples contain primarily cell remnants and only minimal ECM proteins. When hMSC samples were decellularized, the material yield decreased by 95%. By comparison, the decellularization of LF seeded samples decreased yield by approximately 20%. The results indicate that although a significant biological material can be extracted from hMSC seeded foams, the material contains primarily cell remnants and negligible amounts of ECM including collagen.

1-28. (canceled)

29. A method of making a three-dimensional extracellular matrix comprising:
facilitating the production of a scaffold-extracellular matrix composition that includes extracellular matrix on a three-dimensional scaffold comprising a dissolvable polymer; and
contacting the scaffold with a solvent capable of dissolving the dissolvable polymer to produce the three-dimensional extracellular matrix.

30. The method of claim 29, wherein the dissolvable polymer is polyurethane, poly(L-lactic acid), poly(ε-caprolactone), or poly(lactic-co-glycolic acid).

31. The method of claim 29, wherein the step of facilitating the production of the scaffold-extracellular matrix composition comprises:
contacting the scaffold with a liquid extracellular matrix composition to form a scaffold-extracellular matrix mixture; and
treating the scaffold-extracellular matrix mixture to produce the scaffold-extracellular matrix composition.

32. The method of claim 29, wherein the step of facilitating the production of the scaffold-extracellular matrix composition comprises contacting the scaffold with cells to create a cellularized scaffold, and allowing the scaffold-extracellular matrix composition to form from the cellularized scaffold.

33. The method of claim 32, wherein the cells are selected from the group consisting of fibroblasts, stem cells, mesenchymal cells, osteoblasts, chondroblasts, tenocytes and myocytes.

34. The method of claim 32, wherein the step of contacting the scaffold with cells comprises implanting the scaffold into a subject, and wherein the method further comprises harvesting the scaffold-extracellular matrix composition from the subject prior to contacting the scaffold with the solvent.

35. The method of claim 32, further comprising contacting the cells with an agent capable of altering the extracellular matrix composition.

36. The method of claim 35, wherein the agent is selected from a cytokine or a growth factor.

37. The method of claim 32, further comprising subjecting at least one of the cellularized scaffold, the scaffold-extracellular matrix composition or the three-dimensional extracellular matrix to a mechanical stress or a physical stress.

38. The method of claim 29, further comprising removing cells from the three-dimensional extracellular matrix to create a decellularized extracellular matrix.

39. The three-dimensional extracellular matrix of claim 29.

40. The decellularized three-dimensional extracellular matrix of claim 39.

41. A method of generating a tissue in a subject comprising making a three-dimensional extracellular matrix according to the method of claim 29, and implanting the three-dimensional extracellular matrix in the subject, thereby stimulating generation of the tissue in the subject.

42. The method of claim 41, wherein the tissue is selected from the group consisting of larynx, tendon, voice box, ligament, bone, cartilage, soft tissue, nervous tissue, skin, cardiac tissue, and vascular tissue.

43. The method of claim 41, further comprising removing the tissue from the subject.
